(19) United States
(12) Patent Application Publication
(10) Pub. No.: US 2026/0168041 A1
(43) Pub. Date: Jun. 18, 2026

(54) CHARACTERIZING DENGUE VIRUS

(71) Applicant: ILLUMINA, INC., San Diego, CA (US)

(72) Inventor: Jeffrey Koble, San Diego, CA (US)

(21) Appl. No.: 19/425,600

(22) Filed: Dec. 18, 2025

Related U.S. Application Data
(63) Continuation of application No. PCT/US2024/044468, filed on Aug. 29, 2024.
(60) Provisional application No. 63/536,337, filed on Sep. 1, 2023.

(30) Foreign Application Priority Data
Aug. 29, 2024 (WO) PCT/US2024/044468

(51) Int. Cl. C12Q 1/70

(52) U.S. Cl. CPC C12Q 1/701; C12Q 2600/156

(57) ABSTRACT

Provided herein are methods and compositions relating to the characterization of the dengue virus genome. The disclosure provides oligonucleotides primers for the amplification of the dengue virus genome for downstream sequencing, alignment, and identification of variants in the dengue virus genome.

### CROSS-REFERENCE TO RELATED APPLICATION

This application is a bypass continuation of PT/2024/044468, claims the benefit of U.S. Provisional Application No. 63/536,337 dated Sep. 1, 2023, the disclosures of which are incorporated herein by reference in their entireties.

### SEQUENCE LISTING

This application contains a Sequence Listing which has been submitted electronically as an XML file named 35629-0048WO1. The XML file, created on Aug. 23, 2024, is 136,223 bytes in size. The material in the XML file is hereby incorporated by reference in its entirety.

### TECHNICAL FIELD

The present disclosure provides methods and compositions for amplification, sequencing, and characterization of the dengue virus genome.

### BACKGROUND

Viral genomes can accumulate mutations during replication and propagation in a population. Viruses that have RNA as genetic material, e.g., SARS-CoV-2, influenza, chikungunya, and dengue, accumulate mutations at a faster rate than viruses with DNA genomes. Accumulated mutations may confer varying degrees of pathogenicity or transmissibility and become more or less prevalent in a population. Genomic sequencing is useful for identifying variants of a virus in a specimen. Genomic surveillance, including sequencing, can be used by public health authorities to track the spread of viral variants and monitor changes in viral genomes in a population. This information can be used to better understand how circulating variants impact public health. The present disclosure provides methods and compositions that are particularly useful for characterizing the dengue virus genome.

### SUMMARY

The present disclosure relates to methods and compositions for characterizing the dengue virus genome. In particular, the methods and compositions of the disclosure relate to, for example, oligonucleotide primers for amplification of the dengue virus genome, sequencing methods, alignment of sequencing reads to a dengue virus reference genome, and detection and characterization of genomic variants in samples, e.g., biological samples, that may contain dengue virus genome samples. The disclosure provides methods of characterizing a dengue virus genome, including obtaining a nucleic acid of viral origin; obtaining virus-specific oligonucleotide primers (oligonucleotide sequences provided in Table 1 below); amplifying the nucleic acid using the oligonucleotide primers; sequencing the amplification products to produce sequencing reads; aligning the sequencing reads to a reference dengue genome to produce an alignment; and based on the alignment, identifying variants in the dengue virus genome.

In a first aspect, the disclosure provides methods of characterizing a dengue virus genome, the methods including: obtaining a sample including a nucleic acid of viral origin; obtaining a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71; obtaining a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70; amplifying the nucleic acid using the first and the second pluralities of oligonucleotide primers to produce first and second amplification products; sequencing the first and the second amplification products to produce sequencing reads; aligning the sequencing reads to a reference dengue genome to produce an alignment; and, based on the alignment, identifying one or more variants in the dengue virus genome compared to the reference dengue virus genome.

In some embodiments, the nucleic acid is RNA. In some embodiments, the methods further include, before the amplification step, reverse transcribing the RNA to produce cDNA. In some embodiments, the first plurality of oligonucleotide primers are configured for use as pairs, wherein the pairs are selected from the group consisting of SEQ ID NOs: 2 and 4; 5 and 8; 9 and 12; 13 and 16; 17 and 20; 21 and 24; 25 and 28; 29 and 32; 33 and 36; 37 and 40; 41 and 44; 45 and 48; 49 and 52; 53 and 56; 57 and 60; 61 and 64; 65 and 68; and 69 and 71; and the second plurality of oligonucleotide primers are configured for use as pairs, wherein the pairs are selected from the group consisting of SEQ ID NOs: 3 and 6; 7 and 10; 11 and 14; 15 and 18; 19 and 22; 23 and 26; 27 and 30; 31 and 34; 35 and 38; 39 and 42; 43 and 46; 47 and 50; 51 and 54; 55 and 58; 59 and 62; 63 and 66; and 67 and 70.

In some embodiments, the reference dengue genome is about 80%, 85%, 90%, 95%, 96%, 87%, 98%, 99%, or 100% identical to SEQ ID NO: 1. In some embodiments, the sequencing is next generation sequencing (NGS). In some embodiments, the nucleic acid is obtained from a biological sample. In some embodiments, the sample includes blood or serum. In some embodiments, the methods further include, after the alignment step, quantifying a genomic coverage of the sequencing reads. In some embodiments, the genomic coverage is at least 50%, 55%, 60%, 65%, 70%, 75%, 80%, 85%, 95%, 96%, 97%, 98%, 99%, or 100% of the reference dengue genome at a minimum read depth of at least ten reads.

In some embodiments, the RNA is present in the sample in an amount equivalent to 150 copies of the dengue virus genome. In some embodiments, the genomic coverage is at least 70% of the reference dengue genome at a minimum read depth of at least ten reads. In some embodiments, the RNA is present in the sample in an amount equivalent to 500 copies of the dengue virus genome. In some embodiments, the genomic coverage is at least 90% of the reference dengue genome at a minimum read depth of at least ten reads. In some embodiments, the RNA is present in the sample in an amount equivalent to 5000 copies of the dengue virus genome. In some embodiments, the genomic coverage is at least 95% of the reference dengue genome at a minimum read depth of at least ten reads. In some embodiments, the methods further include distinguishing the dengue virus genome from a second nucleic acid of viral origin based on the identified variants.

In another aspect, the disclosure provides methods of detecting dengue virus RNA in a sample, e.g., for diagnostic, research, or surveillance purposes. The methods include: obtaining a sample; isolating RNA from the sample; obtaining a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71; obtaining a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70; reverse transcribing the RNA to produce cDNA amplifying the cDNA using the first and the second pluralities of oligonucleotide primers to produce first and second amplification products; sequencing the first and the second amplification products to produce sequencing reads; quantifying the sequencing reads; and determining, based on the quantity of sequencing reads, a presence or absence of dengue virus RNA in the sample.

In some embodiments, the sample is a biological sample. In some embodiments, the biological sample includes blood or serum. In some embodiments, the sample is an environmental sample. In some embodiments, the environmental sample includes an extract from one or more mosquitos, or a wastewater or air filter sample.

In some embodiments, the first plurality of oligonucleotide primers are configured for use as pairs, wherein the pairs are selected from the group consisting of SEQ ID NOs: 2 and 4; 5 and 8; 9 and 12; 13 and 16; 17 and 20; 21 and 24; 25 and 28; 29 and 32; 33 and 36; 37 and 40; 41 and 44; 45 and 48; 49 and 52; 53 and 56; 57 and 60; 61 and 64; 65 and 68; and 69 and 71; and the second plurality of oligonucleotide primers are configured for use as pairs, wherein the pairs are selected from the group consisting of SEQ ID NOs: 3 and 6; 7 and 10; 11 and 14; 15 and 18; 19 and 22; 23 and 26; 27 and 30; 31 and 34; 35 and 38; 39 and 42; 43 and 46; 47 and 50; 51 and 54; 55 and 58; 59 and 62; 63 and 66; and 67 and 70.

In some embodiments, the sequencing is next generation sequencing (NGS). In some embodiments, the methods further include assembling the sequencing reads to produce a consensus sequence. In some embodiments, the consensus sequence is produced if at least 35 amplicons are detected in the sequencing reads.

In another aspect, the disclosure provides kits including: one or more buffers; a reverse transcriptase; a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71; a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70; a DNA polymerase; and one or more library preparation agents.

The methods and compositions disclosed herein provide for successful amplification of the dengue virus genome for downstream sequencing resulting in high genomic coverage. Compared to previously available methods and compositions, the oligonucleotide primers provided by the disclosure result in increased sequencing coverage of the dengue virus genome. Further, the sets of oligonucleotide primers provided in Table 1 can provide an amplicon sequencing library that identifies variants in the dengue virus genome and is useful for distinguishing between strains of the dengue virus genome.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is an Integrative Genomics Viewer window of genomic sequencing coverage after amplification of a dengue virus genome using the oligonucleotide primers disclosed herein, with an RNA input equivalent to 150, 500, and 5000 copies of the dengue virus genome (top, middle, bottom tracks, respectively). Sequencing reads were aligned to the dengue virus reference genome NCBI Genbank Reference Sequence KM204119.1 (SEQ ID NO: 1).

FIG. 1B is an Integrative Genomics Viewer window of genomic sequencing coverage for three technical replicates after amplification of a dengue virus genome using the oligonucleotide primers disclosed herein, with an RNA input equivalent to 150 copies of the dengue virus genome.

FIG. 1C is an Integrative Genomics Viewer window of genomic sequencing coverage for three technical replicates after amplification of a dengue virus genome using the oligonucleotide primers disclosed herein, with an RNA input equivalent to 500 copies of the dengue virus genome.

FIG. 2A is a plot of genomic sequencing coverage after amplification of a dengue virus genome using previously available oligonucleotide primers, with an RNA input equivalent to 800 copies of the dengue virus genome. Sequencing reads were aligned to the dengue virus reference genome NCBI Reference Sequence NC_001477.1.

FIG. 2B is a plot of genomic sequencing coverage after amplification of a dengue virus genome using previously available oligonucleotide primers, with an RNA input equivalent to 800 copies of the dengue virus genome. Sequencing reads were aligned to the dengue virus reference genome NCBI Reference Sequence MK506262.1.

### DETAILED DESCRIPTION

The disclosure provides an amplicon-based library preparation solution for the sequencing and characterization of the dengue virus genome. The disclosure provides a set of oligonucleotide primers for the amplification of the dengue virus genome.

Sequences of the oligonucleotide primers are provided in Table 1 below. The oligonucleotide primers are designed such that they are divided into two pools with alternate target genome regions, so that neighboring amplicons do not overlap within the same pool. Neighboring amplicons within each primer pool have a gap between each amplicon. The recommended analysis solution for the workflow is the DRAGEN™ Targeted Microbial Application (DTMA)™ implemented in BaseSpace™ for alignment, variant calling, and consensus genome output. Compared to previously available methods and compositions, the oligonucleotide primers provided by the disclosure result in increased sequencing coverage of the dengue virus genome.

The disclosure describes in further detail below the dengue virus; methods of virus surveillance; oligonucleotide primers, PCR amplification, and sequencing; workflow of the methods disclosed herein; and kits for performing the methods disclosed herein.

### Dengue Virus

The dengue virus (DENV) is the cause of dengue fever. It is a mosquito-borne, single positive-stranded RNA virus of the family Flaviviridae, genus Flavivirus. Four serotypes of the virus are known, all of which can cause the full spectrum of disease. The rate of nucleotide substitution for this virus has been estimated to be 6.5×10⁴ per nucleotide per year, a rate similar to other RNA viruses.

The DENV genome is a 10,736 base single-stranded RNA that codes for three structural proteins (capsid protein C, membrane protein M, envelope protein E) and seven nonstructural proteins (NS1, NS2a, NS2b, NS3, NS4a, NS4b, NS5). The DENV genome also includes short noncoding regions on both the 5′ and 3′ ends.

Common names for dengue fever include breakbone fever, vomiting and dandy fever; dengue hemorrhagic fever and dengue shock syndrome. Dengue is found in tropical and subtropical climates worldwide, mostly in urban and semiurban areas. People of all ages who are exposed to infected mosquitoes are at risk for developing dengue fever. The disease occurs most often during the rainy season in tropical countries in Southeast Asia, South Asia, and South America, with high numbers of infected mosquitoes. The virus is transmitted to humans through the bites of infected female mosquitoes, though humans are not capable of transmitting the disease and are not contagious. The incubation period is 3 to 14 days, while the period of the illness is 3-7 days. Signs and symptoms may include severe headache; retro-orbital pain; muscle, joint, and bone pain; macular or maculopapular rash; and minor hemorrhagic manifestations, including petechiae, ecchymosis, purpura, epistaxis, bleeding gums, hematuria, or a positive tourniquet test result. Clinical features, epidemiology, pathogenesis relating to dengue virus infection are reviewed in, for example, Halstead S. Recent advances in understanding dengue. F1000Res. 2019 Jul. 31; 8:F1000 Faculty Rev-1279.

The complete single stranded RNA reference genome for dengue virus is publicly available and accessible at the website of the National Center for Biotechnology Information (NCBI), listed as Genbank Reference Sequence KM204119.1 and provided as SEQ ID NO: 1 below:

(SEQ ID NO: 1)
anttgttagtctnngtggaccgacaagaacagtttcgaatcggaagcttgcttaacgtagttctgacagtt
ttttatttagagagcagatctctgatgaacaaccaacggaaaaagacgggtcgaccgtctttcaatatgct
gaaacgcgcgagaaaccgcgtgtcaactgtttcacagttggcgaagagattctcaaaaggattgctttcag
gccaaggacccatgaaattggtgatggcttttatagcattcctaagatttctagccatacccccaacagca
ggaattttggctagatggggctcattcaagaagaatggagcgattaaagtgctacggggtttcaagaaaga
aatctcaaacatgctgagcataatgaatagaagaaaaagatccgtgaccatgctccttatgctgctgccca
cagccctggcgttccatctgaccacacgagggggagagccgcacatgatagttagcaagcaggaaagagga
aagtcacttttgtttaagacctctgcaggtgtcaacatgtgcacccttattgcgatggatttgggagagtt
atgtgaggacacaatgacttacaaatgcccccggatcactgaggcggaaccagatgacgttgactgttggt
gcaatgccacggacacatgggtgacctatggaacgtgttctcaaactggcgaacaccgacgagacaagcgt
tccgtcgcactggccccacatgtggggcttggtctagaaacaagagccgaaacgtggatgtcctctgaagg
cgcttggaaacaaatacaaaaagtggagacttgggctctgagacacccaggattcacggtaacagccctct
ttctagcacatgccataggaacatccatcacccagaaagggattattttcattttgttgatgctggtaaca
ccatccatggccatgcgatgcgtgggaataggcaacagagacttcgtggaaggactgtcaggagcaacgtg
ggtggatgtggtactggagcatggaagttgcgtcaccaccatggcaaaagataaaccaacattggacattg
aactcttgaagacggaggtcacaaaccctgccgtcctgcgtaaactgtgcattgaagctaaaatatcaaac
accaccaccgattcaagatgtccaacacaaggggaagccacactggtggaagaacaagacgcgaacttcgt
gtgtcgacgaacgtttgtggacagaggctggggcaatggctgtgggcttttcggaaaaggtagcctaataa
cgtgtgctaagttcaagtgtgtgacaaaactggaaggaaagattgttcaatatgagaacttgaaatattca
gtgatagtcaccgtccacactggtgaccagcaccaggtgggaaatgagaccacagaacatggaacaactgc
aaccataacacctcaagctcctacgtcggaaatacagctgaccgactacggagctcttacattggattgct
cacccagaacagggctagactttaatgagatggtgttgttgacaatgaaagaaaaatcatggcttgtccac
aaacaatggtttctagacttaccactgccctggacctcgggagcttcaacaccccaagagacttggaacag
acaagatttgctggttacatttaagacagctcatgcaaagaagcaggaagtagtcgtactaggatcacaag
aaggagcaatgcacactgcgttgaccggagcgacagaaatccaarcgtctggaacgacaacaatttttgca
ggacacttgaaatgtagactaaaaatggacaaactgaccttaaaagggatgtcatatgtgatgtgcacagg
ctcattcaagttagagaaagaagtggctgagacccagcatggaactgttctagtgcaggttaaatacgaag
gaacagatgcaccatgcaagatccccttttcgacccaagatgagaaaggagtaacccagaatgggagattg
ataacagccaaccccatagtcactgacaaagaaaaaccagtcaacattgaggcagaaccaccttttggtga
gagttacatcgtggtaggagcaggtgaaaaagctttgaaactaagctggttcaagaaaggaagcagcatag
ggaaaatgcttgaagcaactgcccgaggagcacgaaggatggccatcctaggagacaccgcatgggacttc
ggttctataggaggagtgttcacgtctgtgggaaaactggtacaccagatttttggaactgcatatggagt
tttgttcagcggtgtttcctggactatgaaaataggaatagggattctgctgacatggctaggattaaatt
caaggagcacgtccctttcgatgacgtgcattgcagttggcatggttacactgtacctaggagtcatggtt
caggcggactcgggatgtgtaattaactggaaaggcagagaactcaaatgtggaagtggcatttttgtcac
caatgaagttcacacttggacagagcaatacaaattccaggccgactccccaaagagactatcagcagcca
ttgggaaggcatgggaagagggtgtgtgtggaattcgatcagccactcgtctcgagaacatcatgtggaag
cagatatcaaatgaactaaaccacatcttacttgaaaatgacatgaaattcacagtggtcgtaggagatgt
tagtgggatcttggcccaaggaaaaaaaatgattgggccacaacccatggaacacaaatactcgtggaaaa
gctggggaaaagccaaaatcataggagcagatgtacagaacaccaccttcattatcgacggcccaaacacc
ccagaatgccctgatgaccaaagagcatggaacatttgggaagttgaggactatggatttggaattttcac
gacaaatatatggttgaaattgcgtgactcctacacccaagtgtgtgacccccggctaatgtcagctgcca
tcaaggacagcaaggcagttcatgccgatatggggtactggatagaaagtgaaaagaacgagacctggaag
ctggcgagagcctccttcatagaagttaagacatgcgtctggccaaaatcccacactctatggagcaatgg
agttttggaaagtgaaatgataatcccaaagatatatggaggaccaatatctcagcacaactacagaccag
gatatttcacacaaacagcagggccgtggcacctaggcaagttggaactagattttgatttgtgtgagggt
accacagttgttgtggatgaacattgtggaaatcgaggaccatctcttagaaccacaacagtcacaggaaa
gataatccatgaatggtgctgtagatcttgtacgctaccccccttacgtttcaaaggagaagacgggtgtt
ggtacggtatggaaatcagaccagtcaaggacaaggaagagaacctagttaagtcattggtctctgcaggg
tcaggagaagtggatagcttttcactaggactgctatgcatatcaataatgatcgaagaggtgatgagatc
cagatggagtagaaaaatgctgatgactggaacactggctgtgttcctccttctcataatgggacaattga
catggaatgatctgatcaggttatgcatcatggttggagccaatgcttcagacaggatggggatgggaaca
acgtacctagctctgatggccacttttaaaatgagaccaatgttcgctgtcgggttattatttcgcagact
aacatctagagaagttcttcttcttacgattggattgagtctggtggcatctgtggagctaccaaattcct
tggaggagctgggggatggacttgcaatgggcatcatgattttaaaattactgactgactttcagtcacat
cagctgtgggctgccctgctgtccttgacatttatcaaaacaactttttcattgcactatgcatggaagac
aatggctatggtactgtcaattgtatctctcttccctttatgcctgtccacgacctctcaaaaaacaacat
ggcttccggtgctgttgggatctcttggatgcaaaccactaaccatgtttcttatagcagaaaacaaaatc
tggggaaggaaaagttggcccctcaatgaaggaatcatggctgttggaatagttagcatcctactaagttc
actcctcaagaatgatgtgccgctagccggcccactaatagctggaggtatgctaatagcatgttatgtta
tatccggaagctcagccgatttatcactggagaaagcggctgaggtctcctgggaagaagaagcagaacac
tctggtgcctcacacaacatactagtggaagtccaagatgatggaaccatgaagataaaagatgaagagag
agatgacacactcaccattctccttaaagcaactctgttggcagtctcaggggtgtacccaatatcaatac
cagcgaccctttttgtgtggtatttttggcagaaaaagaaacagagatcaggagtgctatgggacacacct
agccctccagaagtggaaagagcagttcttgatgatggtatctatagaattctgcaaagaggactgttggg
caggtcccaagtaggagtaggagttttccaagaaggcgtgttccacacaatgtggcatgtcaccaggggag
ctgtcctcatgtatcaagggaagagactggaaccgagttgggccagtgtcaaaaaagacttgatctcatat
ggaggaggttggaggtttcaaggatcctggaacacgggagaagaagtgcaggtgattgctgttgaaccggg
gaaaaaccccaaaaacgtacagacagcgccgggcaccttcaagacccctgaaggcgaagttggagccatag
ccctagattttaaacccggcacatctggatctcccattgtgaacagagagggaaaaatagtaggtctttat
ggaaatggagtggtgacaacaagtggaacctacgtcagtgccatagctcaagctaaagcatcacaagaagg
gcctctaccagagattgaggacgaggtgtttaggaaaagaaacttaacaataatggacctacatccaggat
cggggaaaacaagaagatatcttccagccatagtccgtgaggctataaaaaggaagctgcgtacgctaatc
ttggctcccacaagagttgtcgcttctgaaatggcagaggcgctcaagggaatgccaataaggtatcagac
aacagcagtgaagagtgaacacacaggaaaggagatagttgaccttatgtgccatgccactttcaccatgc
gtctcctgtctcccgtgagagttcccaattacaacatgattatcatggatgaagcacatttcaccgatcca
gccagcatagcggccagagggtacatctcaacccgggtgggcatgggtgaagcagctgcgatcttcatgac
agccactcccccaggatcggtggaggcctttccacagagcaatgcagttatccaagatgaggaaagagaca
ttcctgagagatcatggaactcaggctatgactggatcactgatttcccaggtaaaacagtctggtttgtt
ccaagcattaaatcaggaaatgacattgccaactgtttaagaaagaatgggaaacgggtgatccaattgag
cagaaaaacctttgatactgagtaccagaaaacaaaaaataatgactgggactatgtcgtcacaacagaca
tttccgaaatgggagcaaacttccgagccgacagggtaatagacccaagacggtgtttgaaaccggtaata
ctaaaagatggtccagagcgtgtcattctagccggaccgatgccagtgactgtggccagtgccgcccagag
gagaggaagaattggaaggaaccaaaataaggaaggtgatcagtacatttacatgggacagccttyaaaca
acgatgaggatcacgctcattggacagaagcaaaaatgctccttgacaacataaacacaccagaagggatt
atcccagccctctttgagccggagagagraaaaagtgcagcaatagacggggaatacagactgcggggtga
agcaaggaaaacgttcgtggagctcatgagaagaggagatctacctgtctggctatcctacaaagttgcct
cagaaggcttccagtactctgacagaaagtggtgctttgatggggaaaggaacaaccaggtgttggaggag
aacatggacgtggagatctggacaaaagaaggagaaagaaagaaactacgaccccgctggctggacgccag
aacatactctgacccacaggctctgcgcgagtttaaagagtttgcagcaggaagaagaagcgtctcaggtg
atctaatattagaaatagggaaacttccacaacatttgacgcaaagggcccagaatgctttggacaacctg
gtcatgttgcacaactccgaacaaggaggaaaagcctatagacatgctatggaagaactaccagacaccat
agaaacgttgatgctcctagctttgatagctgtgttaactggtggagtgacgctgttcttcctatcaggaa
gaggcctagggaaaacatctatcggcctactctgcgtgatggcttcaagcgtactgttatgggtggccagt
gtggagccccattggatagcggcctccatcatactggagttctttctgatggtgctgcttattccagagcc
agacagacaacgcactccacaggacaaccagctggcatatgtggtgataggtttgttattcatgatattga
cagtagcagccaatgagatgggattattggaaaccacaaagaaagacctagggattggccatgtggctgtt
gaaaaccaccaccatgccacaatgctggacgtagacttacrtccagcttcagcctggaccctctatgcagt
ggccacaacaattatcactcccatgatgagacacacaattgaaaacacaacggcaaatatttccctgacag
ctattgcaaaccaggcagctatattgatgggacttgacaagggatggccaatatcgaagatggacatagga
gttccactcctcgccttggggtgctattcccaggtgaacccgctgacgctgatagcggcggtattgatgct
agtggctcattacgccataattggacctggactgcaagcaaaagccactagagaagctcaaaaaagaacag
cggccggaataatgaaaaatccaactgtcgacggaattgttgcaatagatttggaccctgtggtttatgat
gcaaaatttgaaaaacagctaggccaaataatgttgttgatactttgcacatcacagattctcttgatgcg
gactacatgggccttgtgtgaatccatcacactggctactggacctctgaccacgctttgggagggatctc
caggaaaattctggaacaccacgatcgcggtgtccatggcaaacattttcaggggaagttatctagcagga
gcaggtctggccttctcattaatgaaatctctaggaggaggtaggagaggtacgggagcccaaggggaaac
actgggagaaaaatggaaaagacagctaaaccaactgagcaagtcagaattcaacacttacaaaaggagtg
ggattatggaggtggatagatctgaagctaaagagggactgaaaagaggagaaacaaccaaacatgcagtg
tcgagaggaacagccaaactgaggtggtttgtggagaggaaccttgtgaagccggaagggaaagtcataga
cctcggttgtggaagaggtggctggtcatattattgtgctgggctgaagaaagtcacagaagtgaaaggat
acacaaaaggaggacctggacatgaggaaccaatcccaatggcgacctatggatggaacctagtaaagcta
cactccgggaaagatgtattctttacaccacctgagaaatgtgacacccttttgtgtgatattggtgagtc
ctctccgaacccaactatagaagaaggaagaacgttacgtgttctaaagatggtggaaccatggctcagag
gaaaccaattttgcataaaaattctaaatccctatatgccgagtgtggtggaaactctggagcaaatgcaa
agaaaacatggaggaatgctagtgcgaaatccactctcaagaaattccacccatgaaatgtactgggtttc
atgtggaacaggaaacattgtgtcagcagtaaacatgacatctagaatgttgctaaatcggttcacaatgg
ctcacaggaagccaacatatgaaagagacgtggacttaggtgctggaacaagacatgtggcagtggaacca
gaggtagccaacctagatatcattggccagaggatagagaatataaaaaatgagcataagtcaacatggca
ttatgatgaggacaatccatacaaaacatgggcctatcatggatcatatgaggttaagccatcaggatcag
cctcatccatggtcaatggcgtggtgagattgctcaccaaaccatgggatgttatccctatggtcacacaa
atagccatgactgacactacaccctttggacaacagagggtgtttaaagagaaagttgacacgcgcacacc
aaaagcaaaacgaggcacagcacaaatcatggaggtgacagccaggtggttatggggttttctttctagaa
acaaaaaaccaagaatctgcacaagagaggagttcacaagaaaagtcaggtcaaacgcagctattggagca
gtgttcgttgatgaaaatcaatggaactcagcaaaagaagcggtggaagatgaacggttctgggaccttgt
gcacagagagagggagctccataaacagggaaaatgtgccacgtgtgtttacaatatgatggggaagagag
agaaaaaattaggagagttcggaaaggcaaaaggaagtcgtgcaatatggtacatgtggttgggagcacgc
tttctagagttcgaagcccttggtttcatgaacgaagatcactggttcagtagagagaattcactcagtgg
agtggaaggagaaggactccacaaacttggatatatactcagagacatatcaaagattccagggggaaata
tgtatgcagatgacacagccggatgggacacaagaataacagaggatgatcttcagaatgaggccaaaatc
actgacatcatggagcccgaacatgccctattggctacgtcaatctttaagctgacctaccaaaacaaggt
ggtaagggtgcagagaccagcaaaaaatggaaccgtgatggatgtcatatccagacgtgaccagagaggaa
gtggacaggtcggaacttatggcttaaacactttcactaacatggaggcccaactaataagacaaatggag
tctgagggaatcttttcacccagcgaattggagaccccaaatttagccgaaagagttctcgactggttgga
aaaacatggcgtcgaaaggctgaaaagaatggcaatcagcggagatgactgtgtggtgaaaccaactgatg
acaggttcgcaacagccttaacagctttgaatgacatgggaaaagtaagaaaagacataccgcaatgggaa
ccttcaaaaggatggaatgattggcaacaagtgcctttttgttcacaccatttccaccagctgatcatgaa
ggatgggagggagatagtggtgccatgccgcaaccaagatgaacttgtgggtagggctagagtatcacaag
gcgccggatggagcctgagagaaactgcatgcctaggcaagtcatatgcacaaatgtggcagctgatgtac
ttccacaggagagacctgagactagcggccaatgccatctgttcagccgttccaattgattgggtcccaac
cagccgcaccacctggtcgatccatgcccatcatcaatggatgacaacagaagacatgttgtcagtgtgga
atagggtttggatagaggaaaacccatggatggaggataaaacccatgtatccagttgggaagatgttcca
tacttaggaaaaagggaagatcagtggtgtggatccctgataggcttaacagcaagggccacctgggccac
taatatacaagtggccataaaccaagtgagaaggcttattgggaatgagaattatctagattacatgacat
caatgaagagattcaagaatgagagtgatcccgaaggggcactctggtaagtcaacacattcacaaaacaa
aggaaaataagaaatcaaacaaggcaagaagtcaggccggattaagccatagtacggtaagagctatgctg
cctgtgagccccgtctaaggacgtaaaatgaagtcaggccggaagccacggtttgagcaaaccgtgctgcc
tgtagctccatcgtggggatgtaaaaacccgggaggctgcaacccatggaagctgtacgcatggggtagca
gactagtggttagaggagacccctcccaaaacacaacgcagcagcggggcccaacaccaggggaagctgta
ccctggtggtaaggactagaggttagaggagaccccccgcacaacaataaacagcatattgacgctgggag
agaccagagatcctgctgtctctacagcatcattccaggcacagaacgccagaaaatggaatggtgctgtt
gaatcaacaggttct

### Virus Detection and Surveillance

Monitoring viruses, both newly emerging viruses and well-established viruses, is an important measure taken to control the spread and impact of viruses in animal and human populations. In some embodiments, the compositions and methods disclosed herein can be used for rapid analysis of many viral samples to identify which isolate or isolates of dengue virus are present in an individual subject and to identify sequence variation between isolates. Early sequence monitoring of many isolates in parallel can be used to rapidly identify isolates and mutations. Some isolates of a given virus, e.g., dengue virus, may have more severe phenotypes than other isolates, for example, higher morbidity or mortality rates and/or greater drug resistance. When a new outbreak occurs, rapid resequencing of isolates from affected individuals can be used to identify individuals infected with isolates known to have more dangerous phenotypes and steps can be taken to aggressively contain the spread of those isolates. For example, a plurality of individuals may be identified as having symptoms of a disease, e.g., dengue infection.

Samples that may contain virus particles can be isolated from each of the affected individuals and re-sequenced to identify variation between samples. The variation may be compared against a reference sequence, e.g., the dengue reference sequence provided herein as SEQ ID NO: 1. In some embodiments, the sequencing data produced by the compositions and methods disclosed herein are compared to a dengue reference sequence that is about 80%, 85%, 90%, 95%, 96%, 87%, 98%, 99%, or 100% identical to SEQ ID NO: 1. In some embodiments, identified variants in the sequencing data are compared against a database of variation and phenotypes associated with these variations to identify individuals who have strains of the virus that are known to be, for example, more easily transmitted than other strains. Aggressive steps may be taken to insure that those individuals infected with the more transmittable strain are isolated so that transmission is limited. Resources may also be allocated to identifying subjects, e.g., people or animals that were likely to have been contacted by individuals with the easily transmitted strain to minimize the spread of strains with more severe phenotypes.

Additional methods of virus surveillance utilizing the amplification and sequencing methods disclosed herein include isolating dengue virus RNA from patient sputum, lung lavage, nasal swabs, air filter samples, wastewater samples, blood bank samples, and blood samples isolated from mosquito populations.

Dengue virus RNA sequencing results generated by the amplification and sequencing methods disclosed herein can be used to report virus sequences in samples for public health and research applications. The amplification and sequencing methods disclosed herein can also be used to perform dengue virus strain typing for monitoring virus evolution and epidemiology in populations of, e.g., humans and mosquitos.

### Oligonucleotide Primers, PCR Amplification, and Sequencing

The disclosure provides an amplicon-based library preparation for the amplification, sequencing, and characterization of the dengue virus genome. The genome of the dengue virus is amplified using a virus-specific oligonucleotide primer set provided herein. The resulting amplicon library can be sequenced, for example, using next-generation sequencing (NGS). In some embodiments, the resulting NGS data are analyzed using the DRAGEN Targeted Microbial Application (DTMA) analysis pipeline implemented in BaseSpace (Illumina, San Diego, CA) for alignment, variant calling, and consensus genome output.

The dengue virus genomic sample can be amplified by a variety of mechanisms, some of which employ the polymerase chain reaction (PCR). See, for example, PCR Technology. Principles and Applications for DNA Amplification (Ed. H.A. Erlich, Freeman Press, NY, N.Y, 1992); PCR Protocols: A Guide to Methods and Applications (Eds. Innis, et al., Academic Press, San Diego, Calif, 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683.202, 4,683,195, 4,800,159 4,965,188, and 5,333,675, each of which is incorporated herein by reference in their entireties for all purposes.

The methods disclosed herein also employ conventional biology methods, software, and systems. Computer software products that are part of the present disclosure typically include computer readable medium having computer-executable instructions for performing the logic steps of the methods disclosed herein. The computer executable instructions may be written in a suitable computer language or combination of several languages. Basic computational biology methods that may be used in the methods disclosed herein are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics. Application in Biological Science and Medicine (CRC Press, London, 2000) and Ouelette and BZevanis Bioinformatics: A Practical Guide for Analysis of Gene and Proteins (Wiley & Sons, Inc., 2″ ed., 2001).

Assays for the amplification of dengue virus genomic samples can be designed by any number of computational primer design tools known in the art. For example, primers for PCR amplification of the dengue virus genome can be designed to tile across the entirety of the genome in overlapping segments.

In some embodiments, as input, a primer design tool can use a FASTA file containing one or more reference genomes, e.g., the dengue virus genome provided herein as SEQ ID NO: 1. In some embodiments, a dengue genome sequence that is about 80%, 85%, 90%, 95%, 96%, 87%, 98%, 99%, or 100% identical to SEQ ID NO: 1 is used as an input for primer design. A desired PCR amplicon length can be specified. A PCR amplicon length can be, for example, 200, 300, 400, 500, 600, 700, 800, 900, 1000, 1100, 1200, 1300, 1400, 1500, 1600, 1700, 1800, 1900, or 2,000 nucleotides. A desired length of overlap between neighboring amplicons can be specified. A desired length of overlap between neighboring amplicons can be, for example, 50, 55, 60, 65, 70, 75, 80, 85, 90, 95, or 100 nucleotides. In some embodiments, oligonucleotide primers are designed such that they are divided into two pools with alternate target genome regions, so that neighboring amplicons do not overlap within the same pool. Neighboring amplicons within each primer pool have a gap between each amplicon.

For RNA viruses a first reverse transcriptase step can be used to generate double stranded DNA from the single stranded RNA. The amplicon-based library preparation disclosed herein for the dengue virus can be designed to re-sequence the approximately 12,000 base sequence published for the dengue virus, provided herein as SEQ ID NO: 1. In some embodiments, the amplicon-based library preparation can be designed to re-sequence an entire genome, such as the genome of the dengue virus; one or more regions of a genome, for example, selected regions of a genome such as those coding for a protein or RNA of interest; a conserved region from multiple genomes, or multiple genomes, such as the genome of a first dengue virus isolate and the genome of a second dengue virus isolate, or the genome of dengue virus and the genome of a different virus.

The synthesis of oligonucleotide primers is well known and routine in the art. The primers may be routinely made through the well-known technique of, for example, solid phase synthesis. Equipment for such synthesis is sold by several vendors including, for example, Applied BioSystems (Foster City, Calif). Any other means for such synthesis known in the art can additionally or alternatively be employed.

In some embodiments, the primers used for amplification hybridize to and amplify genomic DNA, DNA of bacterial plasmids, DNA of DNA viruses or DNA reverse transcribed from RNA of an RNA virus. Methods of amplifying RNA to produce cDNA using reverse transcriptase are well known to those with ordinary skill in the field. In some embodiments, various computer software programs may be used to aid in the design of primers for amplification reactions such as Primer Premier 5 (Premier Biosoft, Palo Alto, Calif); OLIGO Primer Analysis Software (Molecular Biology Insights, Cascade, Colo.); Primer3 (Untergasser A, et al. Primer3—new capabilities and interfaces. Nucleic Acids Res. 2012 August; 40(15):e115); and Primalscheme (Quick J, et al. Multiplex PCR method for MinION and Illumina sequencing of Zika and other virus genomes directly from clinical samples. Nat Protoc. 2017 June; 12(6):1261-1276.) These programs allow the user to input desired hybridization conditions such as melting temperature of a primer-template duplex for example. In some embodiments, an in silico PCR search algorithm, such as (ePCR) is used to analyze primer specificity across a plurality of template sequences which can be readily obtained from public sequence databases such as GenBank for example. An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This algorithm also provides information on primer specificity of the selected primer pairs. In some embodiments, the hybridization conditions applied to the algorithm can limit the results of primer specificity obtained from the algorithm.

In some embodiments, the melting temperature threshold for the primer template duplex is specified to be 35° C., 40° C., 45° C., 50° C., 55° C., 60° C., 65° C., 70° C., or a higher temperature. In some embodiments, the melting temperature for the primer template duplex is specified to be about 60-70° C. In some embodiments the number of acceptable mismatches is specified to be 10, 9, 8, 7, 6, 5, 4, 3, 2, 1, or zero mismatches. In some embodiments, the buffer components and concentrations and primer concentrations may be specified and incorporated into the algorithm, for example, an appropriate primer concentration is about 250 nM and appropriate buffer components are 50 mM sodium or potassium and 1.5 mM Mg.

One with ordinary skill in the art of design of amplification primers will recognize that a given primer need not hybridize to the target nucleic acid with 100% complementarity to effectively prime the synthesis of a complementary nucleic acid strand in an amplification reaction. Moreover, a primer may hybridize over one or more segments such that intervening or adjacent segments are not involved in the hybridization event (e.g., a loop structure or a hairpin structure). The oligonucleotide primers disclosed herein can comprise at least 70%, at least 75%, at least 80%, at least 85%, at least 90%, at least 95%, at least 99%, or 100% sequence identity with any of the primers listed in Table 1. Thus, in some embodiments, an extent of variation of 70% to 100%, or any range therewithin, of the sequence identity is possible relative to the specific primer sequences disclosed in Table 1. Determination of sequence identity is described in the following example: a primer 20 nucleobases in length which is identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid is between about 70% and about 75%. In other embodiments, homology, sequence identity or complementarity, is between about 75% and about 80%. In yet other embodiments, homology, sequence identity, or complementarity, is at least 85%, at least 90%, at least 92%, at least 94%, at least 95%, at least 96%, at least 97%, at least 98%, at least 99% or is 100%. In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range therewithin) sequence identity with the primer sequences specifically disclosed herein.

One with ordinary skill is able to calculate percent sequence identity or percent sequence homology and able to determine the effects of variation of primer sequence identity on the function of the primer in its role in priming synthesis of a complementary strand of nucleic acid for production of an amplification product of a corresponding segment of virus genome. In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, the amplification steps of the methods disclosed herein are performed using one or more pairs of oligonucleotide primers provided in Table 1. The pairs of oligonucleotide primers can be selected from the group consisting of SEQ ID NOs: 2 and 4; SEQ ID NOs: 5 and 8; SEQ ID NOs: 9 and 12; SEQ ID NOs: 13 and 16; SEQ ID NOs: 17 and 20; SEQ ID NOs: 21 and 24; SEQ ID NOs: 25 and 28; SEQ ID NOs: 29 and 32; SEQ ID NOs: 33 and 36; SEQ ID NOs: 37 and 40; SEQ ID NOs: 41 and 44; SEQ ID NOs: 45 and 48; SEQ ID NOs: 49 and 52; SEQ ID NOs: 53 and 56; SEQ ID NOs: 57 and 60; SEQ ID NOs: 61 and 64; SEQ ID NOs: 65 and 68; SEQ ID NOs: 69 and 71; SEQ ID NOs: 3 and 6; SEQ ID NOs: 7 and 10; SEQ ID NOs: 11 and 14; SEQ ID NOs: 15 and 18; SEQ ID NOs: 19 and 22; SEQ ID NOs: 23 and 26; SEQ ID NOs: 27 and 30; SEQ ID NOs: 31 and 34; SEQ ID NOs: 35 and 38; SEQ ID NOs: 39 and 42; SEQ ID NOs: 43 and 46; SEQ ID NOs: 47 and 50; SEQ ID NOs: 51 and 54; SEQ ID NOs: 55 and 58; SEQ ID NOs: 59 and 62; SEQ ID NOs: 63 and 66; and SEQ ID NOs: 67 and 70. In some embodiments, oligonucleotide primers are designed such that they are divided into two pools with a first pool comprising oligonucleotide primer pairs SEQ ID NOs: 2 and 4; SEQ ID NOs: 5 and 8; SEQ ID NOs: 9 and 12; SEQ ID NOs: 13 and 16, SEQ ID NOs: 17 and 20; SEQ ID NOs: 21 and 24; SEQ ID NOs: 25 and 28; SEQ ID NOs: 29 and 32; SEQ ID NOs: 33 and 36; SEQ ID NOs: 37 and 40; SEQ ID NOs: 41 and 44; SEQ ID NOs: 45 and 48; SEQ ID NOs: 49 and 52, SEQ ID NOs: 53 and 56; SEQ ID NOs: 57 and 60; SEQ ID NOs: 61 and 64; SEQ ID NOs: 65 and 68; and SEQ ID NOs: 69 and 71, and a second pool comprising oligonucleotide primer pairs SEQ ID NOs: 3 and 6; SEQ ID NOs: 7 and 10; SEQ ID NOs: 11 and 14; SEQ ID NOs: 15 and 18; SEQ ID NOs: 19 and 22; SEQ ID NOs 23 and 26; SEQ ID NOs: 27 and 30; SEQ ID NOs: 31 and 34; SEQ ID NOs: 35 and 38; SEQ ID NOs: 39 and 42; SEQ ID NOs: 43 and 46; SEQ ID NOs: 47 and 50; SEQ ID NOs: 51 and 54; SEQ ID NOs: 55 and 58; SEQ ID NOs: 59 and 62; SEQ ID NOs: 63 and 66; and SEQ ID NOs: 67 and 70.

TABLE 1
Oligonucleotide Primers for
Amplification of the Dengue Virus Genome

| Oligo- | | | SEQ |
|---|---|---|---|
| nucleotide | Primer | | ID |
| Name | pool | Sequence | NO |
| DENV1- | 1 | GGACCGACAAG | 2 |
| 400 bp_1_LEFT | | AACAGTTTCGA | |
| DENV1- | 2 | TGGAGCGATTA | 3 |
| 400 bp_2_LEFT | | AAGTGCTACGG | |
| DENV1- | 1 | GGAGCATGGTC | 4 |
| 400 bp_1_RIGHT | | ACGGATCTTTT | |
| DENV1- | 1 | CCAGATGACGT | 5 |
| 400 bp_3_LEFT | | TGACTGTTGGT | |
| DENV1- | 2 | AACGCTTGTCT | 6 |
| 400 bp_2_RIGHT | | CGTCGGTGT | |
| DENV1- | 2 | CGTGGGAATAG | 7 |
| 400 bp_4_LEFT | | GCAACAGAGAC | |
| DENV1- | 1 | AACTTCCATGC | 8 |
| 400 bp_3_RIGHT | | TCCAGTACCAC | |
| DENV1- | 1 | TGTGGGCTTTT | 9 |
| 400 bp_5_LEFT | | CGGAAAAGGTA | |
| DENV1- | 2 | TCTCATATTGA | 10 |
| 400 bp_4_RIGHT | | ACAATCTTTCC | |
| | | TTCCAG | |
| DENV1- | 2 | TGGCTTGTCCA | 11 |
| 400 bp_6_LEFT | | CAAACAATGGT | |
| DENV1- | 1 | GTCTGTTCCAA | 12 |
| 400 bp_5_RIGHT | | GTCTCTTGGGG | |
| DENV1- | 1 | GAAAGAAGTGG | 13 |
| 400 bp_7_LEFT | | CTGAGACCCAG | |
| DENV1- | 2 | TCGAAAAGGGG | 14 |
| 400 bp_6_RIGHT | | ATCTTGCATGG | |
| DENV1- | 2 | TTGAAGCAACT | 15 |
| 400 bp_8_LEFT | | GCCCGAGGA | |
| DENV1- | 1 | GCAGTTCCAAA | 16 |
| 400 bp_7_RIGHT | | AATCTGGTGTA | |
| | | CC | |
| DENV1- | 1 | AGAACTCAAAT | 17 |
| 400 bp_9_LEFT | | GTGGAAGTGGC | |
| | | A | |
| DENV1- | 2 | GGAGTCGGCCT | 18 |
| 400 bp_8_RIGHT | | GGAATTTGTAT | |
| DENV1- | 2 | AATACTCGTGG | 19 |
| 400 bp_10_LEFT | | AAAAGCTGGGG | |
| DENV1- | 1 | GGTGTTTGGGC | 20 |
| 400 bp_9_RIGHT | | CGTCGATAAT | |
| DENV1- | 1 | CGAGAGCCTCC | 21 |
| 400 bp_11_LEFT | | TTCATAGAAGT | |
| | | T | |
| DENV1- | 2 | TCTTTGGGATT | 22 |
| 400 bp_10_RIGHT | | ATCATTTCACT | |
| | | TTCCA | |
| DENV1- | 2 | CCCCCTTACGT | 23 |
| 400 bp_12_LEFT | | TTCAAAGGAGA | |
| DENV1- | 1 | ACTTAACTAGG | 24 |
| 400 bp_11_RIGHT | | TTCTCTTCCTT | |
| | | GTCC | |
| DENV1- | 1 | TGGGAACAACG | 25 |
| 400 bp_13_LEFT | | TACCTAGCTCT | |
| DENV1- | 2 | TGTTAGTCTGC | 26 |
| 400 bp_12_RIGHT | | GAAATAATAAC | |
| | | CCG | |
| DENV1- | 2 | TCTTCCCTTTA | 27 |
| 400 bp_14_LEFT | | TGCCTGTCCAC | |
| DENV1- | 1 | TGGTTTGCATC | 28 |
| 400 bp_13_RIGHT | | CAAGAGATCCC | |
| DENV1- | 1 | CTGAGGTCTCC | 29 |
| 400 bp_15_LEFT | | TGGGAAGAAGA | |
| DENV1- | 2 | CTCTTCATCTT | 30 |
| 400 bp_14_RIGHT | | TTATCTTCATG | |
| | | GTTCCA | |
| DENV1- | 2 | GGTCCCAAGTA | 31 |
| 400 bp_16_LEFT | | GGAGTAGGAGT | |
| DENV1- | 1 | GATACATGAGG | 32 |
| 400 bp_15_RIGHT | | ACAGCTCCCCT | |
| DENV1- | 1 | TCCCATTGTGA | 33 |
| 400 bp_17_LEFT | | ACAGAGAGGGA | |
| DENV1- | 2 | GCTATGGCACT | 34 |
| 400 bp_16_RIGHT | | GACGTAGGTTC | |
| DENV1- | 2 | ACAGCAGTGAA | 35 |
| 400 bp_18_LEFT | | GAGTGAACACA | |
| DENV1- | 1 | GACAGGAGACG | 36 |
| 400 bp_17_RIGHT | | CATGGTGAAAG | |
| DENV1- | 1 | TGGAACTCAGG | 37 |
| 400 bp_19_LEFT | | CTATGACTGGA | |
| DENV1- | 2 | ACAGTTGGCAA | 38 |
| 400 bp_18_RIGHT | | TGTCATTTCCT | |
| | | G | |
| DENV1- | 2 | TGTCATTCTAG | 39 |
| 400 bp_20_LEFT | | CCGGACCGAT | |
| DENV1- | 1 | AGGCTGTCCCA | 40 |
| 400 bp_19_RIGHT | | TGTAAATGTAC | |
| | | TG | |
| DENV1- | 1 | CCTCAGAAGGC | 41 |
| 400 bp_21_LEFT | | TTCCAGTACTC | |
| DENV1- | 2 | CCATGTTCTCC | 42 |
| 400 bp_20_RIGHT | | TCCAACACCTG | |
| DENV1- | 2 | AGGAAAAGCCT | 43 |
| 400 bp_22_LEFT | | ATAGACATGCT | |
| | | ATGG | |
| DENV1- | 1 | TCCACCAGTTA | 44 |
| 400 bp_21_RIGHT | | ACACAGCTATC | |
| | | A | |
| DENV1- | 1 | ACAGTAGCAGC | 45 |
| 400 bp_23_LEFT | | CAATGAGATGG | |
| DENV1- | 2 | GTGGTTTTCAA | 46 |
| 400 bp_22_RIGHT | | CAGCCACATGG | |
| DENV1- | 2 | CGAAGATGGAC | 47 |
| 400 bp_24_LEFT | | ATAGGAGTTCC | |
| | | AC | |
| DENV1- | 1 | GCTTGCAGTCC | 48 |
| 400 bp_23_RIGHT | | AGGTCCAATTA | |
| DENV1- | 1 | ATGGGCCTTGT | 49 |
| 400 bp_25_LEFT | | GTGAATCCATC | |
| DENV1- | 2 | GCGATCGTGGT | 50 |
| 400 bp_24_RIGHT | | GTTCCAGAATT | |
| DENV1- | 2 | AGGGACTGAAA | 51 |
| 400 bp_26_LEFT | | AGAGGAGAAAC | |
| | | A | |
| DENV1- | 1 | GCTTCACAAGG | 52 |
| 400 bp_25_RIGHT | | TTCCTCTCCAC | |
| DENV1- | 1 | GAGTCCTCTCC | 53 |
| 400 bp_27_LEFT | | GAACCCAACTA | |
| DENV1- | 2 | ATTGGTTTCCT | 54 |
| 400 bp_26_RIGHT | | CTGAGCCATGG | |
| DENV1- | 2 | AAGAGACGTGG | 55 |
| 400 bp_28_LEFT | | ACTTAGGTGCT | |
| DENV1- | 1 | TCCTCTGGCCA | 56 |
| 400 bp_27_RIGHT | | ATGATATCTAG | |
| | | GT | |
| DENV1- | 1 | AAAGAGAAAGT | 57 |
| 400 bp_29_LEFT | | TGACACGCGCA | |
| DENV1- | 2 | AGAAAACCCCA | 58 |
| 400 bp_28_RIGHT | | TAACCACCTGG | |
| DENV1- | 2 | AGAGTTCGGAA | 59 |
| 400 bp_30_LEFT | | AGGCAAAAGGA | |
| DENV1- | 1 | ACCAAGGGCTT | 60 |
| 400 bp_29_RIGHT | | CGAACTCTAGA | |
| DENV1- | 1 | TGGCTACGTCA | 61 |
| 400 bp_31_LEFT | | ATCTTTAAGCT | |
| | | GA | |
| DENV1- | 2 | TGACATCCATC | 62 |
| 400 bp_30_RIGHT | | ACGGTTCCATT | |
| DENV1- | 2 | AACTGATGACA | 63 |
| 400 bp_32_LEFT | | GGTTCGCAACA | |
| DENV1- | 1 | TGAAGGTTCCC | 64 |
| 400 bp_31_RIGHT | | ATTGCGGTATG | |
| DENV1- | 1 | ACCTGAGACTA | 65 |
| 400 bp_33_LEFT | | GCGGCCAAT | |
| DENV1- | 2 | TTGATGATGGG | 66 |
| 400 bp_32_RIGHT | | CATGGATCGAC | |
| DENV1- | 2 | GGGAATGAGAA | 67 |
| 400 bp_34_LEFT | | TTATCTAGATT | |
| | | ACATGACA | |
| DENV1- | 1 | TGTGTTGACTT | 68 |
| 400 bp_33_RIGHT | | ACCAGAGTGCC | |
| DENV1- | 1 | GGCCGGATTAA | 69 |
| 400 bp_35_LEFT | | GCCATAGTACG | |
| DENV1- | 2 | TCTAACCTCTA | 70 |
| 400 bp_34_RIGHT | | GTCCTTACCAC | |
| | | CA | |
| DENV1- | 1 | CAGCACCATTC | 71 |
| 400 bp_35_RIGHT | | CATTTTCTGGC | |

### Workflow

The workflow of the amplicon-based library preparation methods disclosed herein can include or consist of the following procedures: dengue virus RNA extraction, cDNA synthesis, target amplification, library preparation, library pooling, sequencing, and analysis.

Dengue virus RNA can be extracted from a biological sample by any means known in the art. A biological sample can be, for example, blood or serum extracted from a patient. In some embodiments, dengue virus RNA is extracted from patient, blood, serum, sputum, lung lavage, nasal swabs. In some embodiments, dengue virus RNA is extracted from environmental sources including air filter samples and wastewater samples. In some embodiments, dengue virus RNA is extracted from sources of blood including blood banks, or is isolated from mosquito populations that may be carrying the virus. Commercially available methods of RNA extraction include, for example, Quick-DNA/RNA Viral MagBead Kit (Zymo Research, #R2141) or QIAamp Viral RNA Mini Kit (Qiagen, part #52906).

In some embodiments, the following steps of the workflow are performed with a dengue virus RNA input equivalent to 100 copies of the dengue virus genome, 200 copies, 300 copies, 400 copies, 500 copies, 600 copies, 700 copies, 800 copies, 900 copies, 1000 copies, 2000 copies, 3000 copies, 4000 copies, or 5000 or more copies.

DNA complementary to the dengue RNA (i.e., cDNA) can be reverse transcribed by reverse transcriptase with random hexamers. Next, the dengue virus genome present in the sample can be amplified using two separate PCR reactions that are then pooled together. In some embodiments, one PCR reaction utilizes oligonucleotide primers designated “Primer Pool 1” in Table 1, and the other PCR reaction utilizes oligonucleotide primers designated “Primer Pool 2” in Table 1. In some embodiments, the pooled amplified fragments undergo tagmentation to further fragment and tag amplicons with adapter sequences. Post-tagmentation yield can be normalized due to saturation of the bead-linked transposome by typical amplicon inputs. The adapter-tagged amplicons can undergo a second round of PCR amplification using a PCR master mix and unique index adapters. After amplification, indexed libraries can be pooled and cleaned using purification beads.

The pooled library product can be quantified using a fluorescent dye with concentration determined by comparison to a DNA standard curve. The pooled library product can be sequenced by any number of commercially available sequencing platforms. In some embodiments, pooled libraries are clustered onto a flow cell, and then sequenced using sequencing by synthesis (SBS) chemistry on, for example, the NovaSeq 6000 Sequencing System using the NovaSeq Xp S4 and SP flow cells, NextSeq 500 System, NextSeq 550 System, NextSeq 550Dx Instrument in RUO mode, or NextSeq 2000 System. The amplification and sequencing workflow disclosed herein can be scaled up or down to accommodate different numbers of samples. For examples, 1536 to 3072 results can be processed on the NovaSeq 6000 system in 12 hours using two SP or S4 reagent kits, or 384 results in 12 hours using the NextSeq 2000 or the NextSeq 500/550/550Dx (in RUO mode) HO reagent kit.

SBS chemistry uses a reversible-terminator method to detect single, fluorescently labeled deoxynucleotide triphosphate (dNTP) bases as they are incorporated into growing DNA strands. During each sequencing cycle, a single dNTP is added to the nucleic acid chain. The dNTP label serves as a terminator for polymerization. After each dNTP incorporation, the fluorescent dye is imaged to identify the base, and then cleaved to allow incorporation of the next nucleotide. Four reversible terminator-bound dNTPs (A, G, T, and C) are present as single, separate molecules. As a result, natural competition minimizes incorporation bias. During the primary analysis, base calls are made directly from signal intensity measurements during each sequencing cycle, resulting in base-by-base sequencing. A quality score is assigned to each base call.

In some embodiments, the Illumina® DRAGEN® Pipeline analyzes sequencing results to detect the presence of dengue virus RNA in each sample. As a quality control feature, an internal control consisting of, for example, one or more human mRNA targets can be included in every sample. Analysis can be performed locally using the Illumina DRAGEN or on BaseSpace® Sequence Hub. In some embodiments, the Illumina DRAGEN Pipeline performs small variant calling and generates a consensus sequence in FASTA format. Analysis can include a quantification of sequencing coverage depth. Sequencing coverage depth refers to the average number of sequencing reads that align to, or cover, each base in a sequenced sample. The Lander/Waterman equation is a method for calculating coverage (C) based on read length (L), number of reads (N), and haploid genome length (G): C=LN/G.

Analysis can include a quantification of genomic coverage. Genomic coverage refers to the breadth of coverage of a target genome, which is defined as the percentage of target bases that are sequenced a given number of times. For example, a genome sequencing study may sequence a genome to 30× average depth and achieve a 95% breadth of coverage of the reference genome at a minimum depth of ten reads. In some embodiments, the methods disclosed herein yield a genomic coverage of 80%, 85%, 95%, 96%, 97%, 98%, 99%, or 100% of the dengue virus genome at a minimum read depth of ten reads.

In some embodiments, when amplification is successful and sequencing reads are generated, a consensus sequence is generated from the sequencing reads. In some embodiments, a contig is assembled from the sequencing reads, wherein the sequencing reads overlap in a way that provides a contiguous representation of the dengue virus genome. In some embodiments, a consensus sequence is generated and reported when at least 30, 31, 32, 33, 34, 35, 36, 37, 38, 39, or 40 different amplicons are detected in the sequencing reads.

### Kits

The present disclosure also provides kits for carrying out the methods described herein. In some embodiments, the kit comprises a sufficient quantity of one or more primer pairs, e.g., one or more of the primer pairs provided in Table 1, to perform an amplification reaction on a DNA reverse transcribed from the dengue virus genome for downstream sequencing.

In some embodiments, the kit comprises a sufficient quantity of reverse transcriptase, a DNA polymerase, suitable nucleoside triphosphates (e.g., dNTPs), a DNA ligase, and/or reaction buffer, or any combination thereof, for the amplification processes described above. A kit can further include instructions pertinent for the particular embodiment of the kit, such as instructions describing the primer pairs and amplification conditions for operation of the methods described herein. A kit can also comprise amplification reaction containers such as microcentrifuge tubes and the like. A kit can also comprise reagents or other materials for isolating dengue virus RNA or identifying resulting amplicons from amplification, including, for example, detergents, solvents, and/or ion exchange resins, which may be linked to magnetic beads. In some embodiments, the kit includes a computer program stored on a computer formatted medium (such as a compact disk or portable USB disk drive, for example) comprising instructions that direct a processor to analyze data obtained from the use of the primer pairs disclosed herein. In some embodiments, the kits of the present disclosure contain all of the reagents sufficient to carry out one or more of the methods described herein.

In some embodiments, the kit further comprises one or more library preparation agents. Library preparation agents can include beads, buffers (e.g., elution buffers, reaction buffers, tagmentation buffers) and reaction mixes (e.g., mixes including enzymes, polymerases, transcriptases, reverse transcriptases).

In some embodiments, the kit comprises one or more of Illumina® Tune Beads and Stop Tagment Buffer 2 HT. In some embodiments, the kit further comprises one or more of enrichment bead-linked transposomes (BLT), elution buffer, resuspension buffer, and tagmentation wash buffer. In some embodiments, the kit further comprises one or more of elution prime fragment 3HC mix, enhanced PCR mix, first strand mix, Illumina PCR mix, a reverse transcriptase, and tagmentation buffer 1. In some embodiments, the kit further comprises a positive control RNA sample.

### EXAMPLES

The invention is further described in the following examples, which do not limit the scope of the invention described in the claims.

### Example 1: Amplification and Sequencing of the Dengue Virus Genome

Oligonucleotide primers listed in Table 1 were computationally designed using a software tool with NCBI Genbank Reference Sequence KM204119.1 as the template genome for primer design. After we designed the primers, DNA oligonucleotides were synthesized according to the design, normalized to 100 M, pooled into two primer pools as described in Table 1, and diluted to 10 M for each pool to generate two overlapping sets of amplicons. RNA input equivalent to 150, 500, and 5000 copies of the dengue virus genome was used for three separate amplification and sequencing experiments. Three technical replicates were performed for each experiment. After amplification, amplicon libraries were denatured and diluted from a pooled library according to the Illumina® NextSeq® 500 and 550 Sequencing Systems Denature and Dilute Libraries Guide (15048776). Libraries were sequenced on the Illumina® NextSeq® 550 instrument at 2×149 base pair read length, unless stated otherwise, and normalized to 1M paired-end read depth based on current sequencing recommendations. Analysis was executed using the DRAGEN® Viral Lineage App v0.4.0.

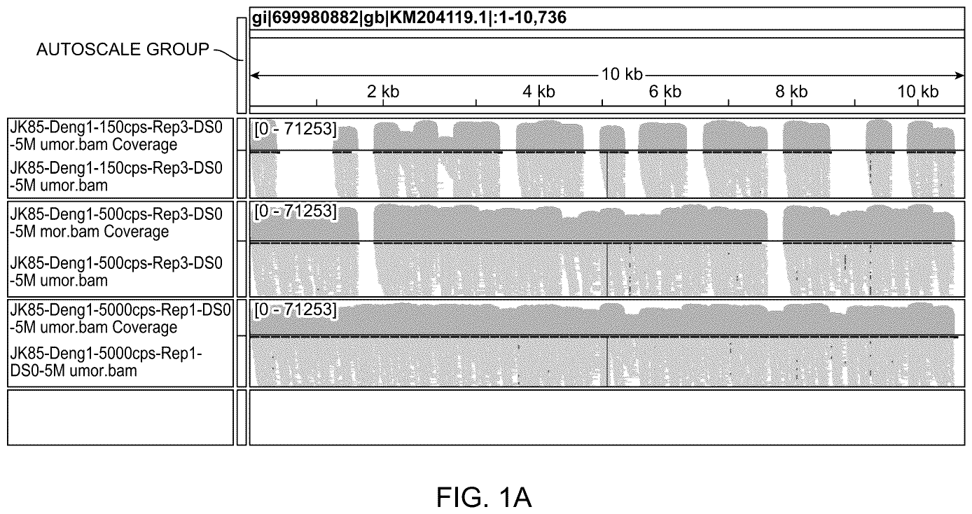

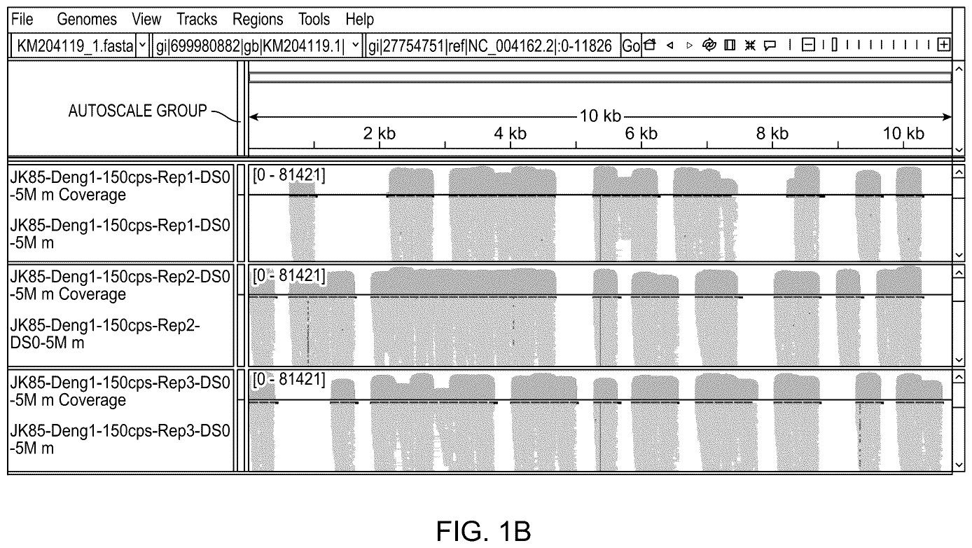

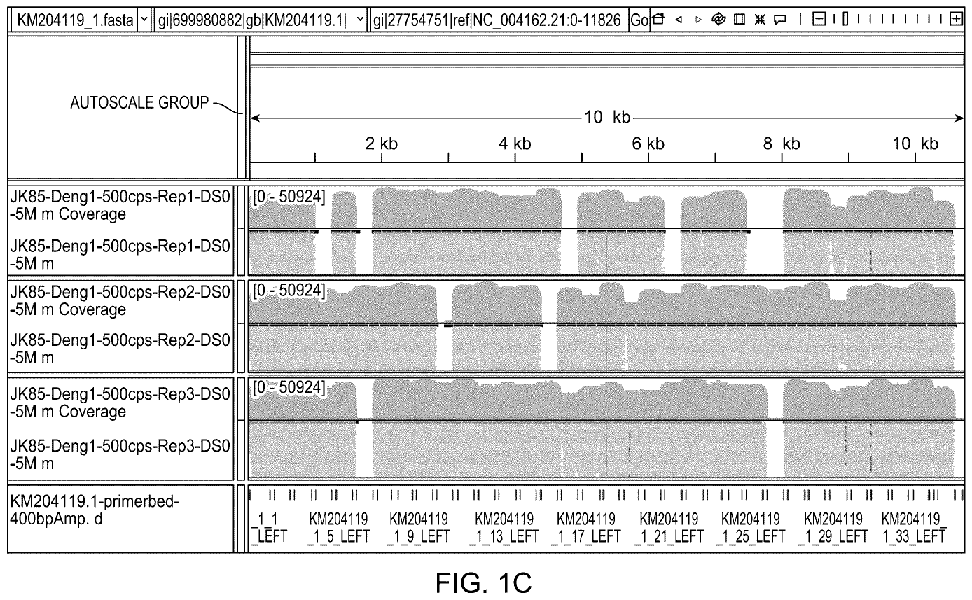

The results of these amplification and sequencing experiments are shown in FIG. 1A. FIG. 1A shows an Integrative Genomics Viewer (Robinson J T, et al. Integrative genomics viewer. Nat Biotechnol. 2011 January; 29(1):24-6. doi: 10.1038/nbt.1754. PMID: 21221095; PMCID: PMC3346182) window of genomic coverage data from 150, 500, and 5000 viral copy input amplified using the oligonucleotide primers provided in Table 1. A track for each amplification and sequencing experiment is displayed. FIG. 1B shows an Integrative Genomics Viewer window of genomic coverage data for the three replicates of the 150 viral copy input experiment. Median genomic coverage at read depth of at least 10× for RNA input equivalent to 150 copies of the dengue virus genome was 70%. FIG. 1C shows an Integrative Genomics Viewer window of genomic coverage data for the three replicates of the 500 viral copy input experiment. Median genomic coverage at read depth of at least 10× for RNA input equivalent to 500 copies of the dengue virus genome was 94%. Median genomic coverage at read depth of at least 10× for RNA input equivalent to 5000 copies of the dengue virus genome was 99%.

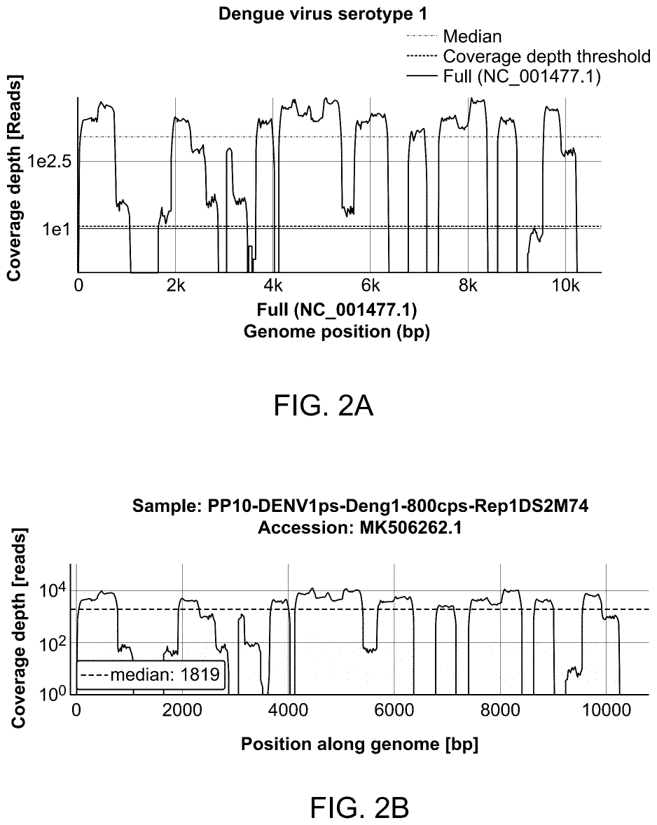

FIGS. 2A-2B show the results of a comparative test using previously available oligonucleotide primers. Amplification and sequencing experiment similar to those described above were performed using the set of previously available oligonucleotide primers. The previously available oligonucleotide primers used in this experiment are provided in Table 2 below. These primers were used to amplify a DENV1 (KM204119.1) dengue virus genome sample. FIG. 2A shows a plot of genomic coverage data from 800 viral copy input using the previously available oligonucleotide primers, with the sequencing reads aligned to reference genome NC_001477.1. The plot in FIG. 2A shows 72.900 coverage with those amplicons aligned to reference genome NC_001477.1. FIG. 2B shows a plot of genomic coverage data from 800 viral copy input using the previously available oligonucleotide primers, with the sequencing reads aligned to reference genome MK506262.1. FIG. 2B shows 74.15% coverage with those amplicons aligned to reference genome MK506262.1.

TABLE 2
Previously Available Oligonucleotide Primers

| | | | SEQ |
|---|---|---|---|
| | | Primer | ID |
| Primer Name | Sequence (5′-3′) | pool | NO |
| DENV1_1_LEFT2 | ACGTGGACCGACAAGAACAG | 1 | 72 |
| DENV1_1_RIGHT | ACTATCATGTGCGGCTCTCC | 1 | 73 |
| DENV1_2_LEFT | TGACCATGCTCCTTATGCTG | 2 | 74 |
| DENV1_2_RIGHT | CAAGCGCCTTCAGAGGACAT | 2 | 75 |
| DENV1_3_LEFT | CACCGACGAGACAAACGTTC | 1 | 76 |
| DENV1_3_RIGHT | AGGGTTTGTGACTTCCGTCT | 1 | 77 |
| | T | | |
| DENV1_4_LEFT | GGAAGTTGCGTCACCACCAT | 2 | 78 |
| DENV1_4_RIGHT | CCATGTTCTGTGCTTTCATT | 2 | 79 |
| | TCC | | |
| DENV1_5_LEFT | AAACTGGAAGGAAAGATAGT | 1 | 80 |
| | TCAATATGA | | |
| DENV1_5_RIGHT | AGTGTGCATTGCTCCTTCTT | 1 | 81 |
| | GT | | |
| DENV1_6_LEFT | TGCTGGTGACATTTAAGACA | 2 | 82 |
| | GC | | |
| DENV1_6_RIGHT | TGTTGACTGGTTTTTCTTTG | 2 | 83 |
| | TCAGT | | |
| DENV1_7_LEFT | GAAGGAACAGATGCACCATG | 1 | 84 |
| | C | | |
| DENV1_7_RIGHT | CCATGTCAGCAGAACCCCTA | 1 | 85 |
| | TTCC | | |
| DENV1_8_LEFT | ACACCAGATTTTTGGAACTG | 2 | 86 |
| | CAT | | |
| DENV1_8_RIGHT | TCATTTGATATTTGCTTCCA | 2 | 87 |
| | CATGATGT | | |
| DENV1_9_LEFT | TGAAGTTCACACTTGGACAG | 1 | 88 |
| | AGC | | |
| DENV1_9_RIGHT | ACTTCCCAAATGTTCCATGC | 1 | 89 |
| | TCT | | |
| DENV1_10_LEFT | GAAAAGCTGGGGAAAGGCTA | 2 | 90 |
| | AA | | |
| DENV1_10_RIGHT | TGTGCTGAGATATTGGTCCT | 2 | 91 |
| | CC | | |
| DENV1_11_LEFT | CCCACACTCTATGGAGCAAT | 1 | 92 |
| | GG | | |
| DENV1_11_RIGHT | ACTTCCCCTGACCCTGCAGA | 1 | 93 |
| DENV1_12_LEFT | ACAGGAAAGATAATTCATGA | 2 | 94 |
| | ATGGTG | | |
| DENV1_12_RIGHT | AACATTGGTCTCATTTTAAA | 2 | 95 |
| | AGTGGC | | |
| DENV1_13_LEFT | TATGCATCATGGTTGGAGCC | 1 | 96 |
| | AA | | |
| DENV1_13_RIGHT | TTTTTGGGAGGTCGTGGACA | 1 | 97 |
| | G | | |
| DENV1_14_LEFT | GCAATGGGCATTATGATTTT | 2 | 98 |
| | AAAAT | | |
| DENV1_14_RIGHT | GCTATTAGCATGCCTCCAGC | 2 | 99 |
| | TA | | |
| DENV1_15_LEFT | ATCTGGGGAAGGAAAAGTTG | 1 | 10 |
| | GC | | |
| DENV1_15_RIGHT | CCAGATCTCTGTTTCTTTTT | 1 | 10 |
| | CTGCC | | |
| DENV1_16_LEFT | AAGATAAAGGATGAAGAGAG | 2 | 10 |
| | AGATGACAC | | |
| DENV1_16_RIGHT | TCTTCTCCCGTGTTCCAGGA | 2 | 10 |
| | TC | | |
| DENV1_17_LEFT | TGTATCAAGGGAAGAGACTG | 1 | 10 |
| | GA | | |
| DENV1_17_RIGHT | ATCCTGGATGTAGGTCCATT | 1 | 10 |
| | ATTGT | | |
| DENV1_18_LEFT | GCTCAAGCTAAAGCATCACA | 2 | 10 |
| | AGA | | |
| DENV1_18_RIGHT | TACCCTCTGGCCGCTATGCT | 2 | 10 |
| DENV1_19_LEFT | TCTCCCGTGAGAGTTCCCAA | 1 | 10 |
| DENV1_19_RIGHT | GTGACGACGTAGTCCCAGTC | 1 | 10 |
| | GT | | |
| DENV1_20_LEFT | AAACGGGTGATTCAATTGAG | 2 | 11 |
| | CA | | |
| DENV1_20_RIGHT | GCTCGAAGAGGGCTGGGATA | 2 | 11 |
| | AT | | |
| DENV1_21_LEFT | CACGCTCACTGGACAGAAGC | 1 | 11 |
| | A | | |
| DENV1_21_RIGHT | TCTTCTTCCTGCTGCAAACT | 1 | 11 |
| | CT | | |
| DENV1_22_LEFT | CTGACAGAAGATGGTGCTTT | 2 | 11 |
| | GA | | |
| DENV1_22_RIGHT | GTCACTCCACCAGTTAACAC | 2 | 11 |
| | A | | |
| DENV1_23_LEFT | TCCGAACAAGGAGGAAGAGC | 1 | 11 |
| | C | | |
| DENV1_23_RIGHT | CCCTAAGTCTTTCTTTGTGG | 1 | 11 |
| | TT | | |
| DENV1_24_LEFT | GCTGGCATATGTGGTGATAG | 2 | 11 |
| | GT | | |
| DENV1_24_RIGHT | CATCAATACCGCCGCTGTCA | 2 | 11 |
| DENV1_25_LEFT | GGACATAGGAGTTCCACTTC | 1 | 12 |
| | TCG | | |
| DENV1_25_RIGHT | GCTATCGTGGTGTTCCAAAA | 1 | 12 |
| | TTT | | |
| DENV1_26_LEFT | ATGGGCCTTGTGCGAATCCA | 2 | 12 |
| | TC | | |
| DENV1_26_RIGHT | GGTTTCACAAGGTTCCTCTC | 2 | 12 |
| | CA | | |
| DENV1_27_LEFT | AAGAGGAGAAACAACCAAAC | 1 | 12 |
| | ATG | | |
| DENV1_27_RIGHT | TTTCCTCTGAGCCATGGTTC | 1 | 12 |
| | CA | | |
| DENV1_28_LEFT | GTGTGATATTGGTGAGTCCT | 2 | 12 |
| | CTCC | | |
| DENV1_28_RIGHT | TCTGGCCAATGATATCTAGG | 2 | 12 |
| | TTGG | | |
| DENV1_29_LEFT | ACAATGGCTCACAGGAAACC | 1 | 12 |
| DENV1_29_RIGHT | CACTTGGCTGTCACCTCCAT | 1 | 12 |
| DENV1_30_LEFT | CTACACCCTTTGGACAACAG | 2 | 13 |
| | AG | | |
| DENV1_30_RIGHT | GCTCCCAACCACATGTACCA | 2 | 13 |
| | TA | | |
| DENV1_31_LEFT | TGTGTTTACAACATGATGGG | 1 | 13 |
| | GAA | | |
| DENV1_31_RIGHT | TTTTTGCTGGTCTCTGTACC | 1 | 13 |
| | C | | |
| DENV1_32_LEFT | AGAATGAGGCCAAAATTACT | 2 | 13 |
| | GACA | | |
| DENV1_32_RIGHT | TCAGAGCTGTTAAGGCTGTT | 2 | 13 |
| | GC | | |
| DENV1_33_LEFT | TGAAAAGAATGGCAATCAGC | 1 | 13 |
| | GG | | |
| DENV1_33_RIGHT | AACTGGAACGGCTGAACAGA | 1 | 13 |
| | T | | |
| DENV1_34_LEFT | TATGCACAAATGTGGCAGCT | 2 | 13 |
| | GA | | |
| DENV1_34_RIGHT | TCTTGAATCTCTTCATTGAT | 2 | 13 |
| | GTCATGT | | |
| DENV1_35_LEFT | CTGGGCCACCAACATACAAG | 1 | 14 |
| | T | | |
| DENV1_35_RIGHT | AGGGGTCTCCTCTAACCACT | 1 | 14 |
| | AG | | |

These results demonstrate that the sets of oligonucleotide primers provided in Table 1 can successfully amplify the dengue virus genome for downstream sequencing resulting in high genomic coverage. Compared to the previously available oligonucleotide primers tested, the oligonucleotide primers provided in Table 1 result in increased sequencing coverage of the dengue virus genome.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of characterizing a dengue virus genome, the method comprising:
obtaining a sample comprising a nucleic acid of viral origin;
obtaining a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71;
obtaining a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70;
amplifying the nucleic acid using the first and the second pluralities of oligonucleotide primers to produce first and second amplification products;
sequencing the first and the second amplification products to produce sequencing reads;
aligning the sequencing reads to a reference dengue genome to produce an alignment; and
based on the alignment, identifying one or more variants in the dengue virus genome compared to the reference dengue virus genome.

2. The method of claim 1, wherein the nucleic acid is RNA.

3. The method of claim 2, further comprising, before the amplification step, reverse transcribing the RNA to produce cDNA.

4. The method of claim 1, wherein the first plurality of oligonucleotide primers are configured for use as pairs, wherein the pairs are selected from the group consisting of SEQ ID NOs: 2 and 4; 5 and 8; 9 and 12; 13 and 16; 17 and 20; 21 and 24; 25 and 28; 29 and 32; 33 and 36; 37 and 40; 41 and 44; 45 and 48; 49 and 52; 53 and 56; 57 and 60; 61 and 64; 65 and 68; and 69 and 71; and
the second plurality of oligonucleotide primers are configured for use as pairs, wherein the pairs are selected from the group consisting of SEQ ID NOs: 3 and 6; 7 and 10; 11 and 14; 15 and 18; 19 and 22; 23 and 26; 27 and 30; 31 and 34; 35 and 38; 39 and 42; 43 and 46; 47 and 50; 51 and 54; 55 and 58; 59 and 62; 63 and 66; and 67 and 70.

5. The method of claim 1, wherein the reference dengue genome is about 80%, 85%, 90%, 95%, 96%, 87%, 98%, 99%, or 100% identical to SEQ ID NO: 1.

6. The method of claim 1, wherein the sequencing is next generation sequencing (NGS).

7. The method of claim 1, wherein the nucleic acid is obtained from a biological sample.

8. The method of claim 7, wherein the sample comprises blood or serum.

9. The method of claim 1, further comprising, after the alignment step, quantifying a genomic coverage of the sequencing reads.

10. The method of claim 9, wherein the genomic coverage is at least 50%, 55%, 60%, 65%, 70%, 75%, 80%, 85%, 95%, 96%, 97%, 98%, 99%, or 100% of the reference dengue genome at a minimum read depth of at least ten reads.

11. The method of claim 2, wherein the RNA is present in the sample in an amount equivalent to 150 copies of the dengue virus genome.

12. The method of claim 11, wherein the genomic coverage is at least 70% of the reference dengue genome at a minimum read depth of at least ten reads.

13. The method of claim 2, wherein the RNA is present in the sample in an amount equivalent to 500 copies of the dengue virus genome.

14. The method of claim 13, wherein the genomic coverage is at least 90% of the reference dengue genome at a minimum read depth of at least ten reads.

15. The method of claim 2, wherein the RNA is present in the sample in an amount equivalent to 5000 copies of the dengue virus genome.

16. The method of claim 15, wherein the genomic coverage is at least 95% of the reference dengue genome at a minimum read depth of at least ten reads.

17. The method of claim 1, further comprising distinguishing the dengue virus genome from a second nucleic acid of viral origin based on the identified variants.

18. A method of detecting dengue virus RNA in a sample, the method comprising:
obtaining a sample;
isolating RNA from the sample;
obtaining a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71;
obtaining a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70;
reverse transcribing the RNA to produce cDNA
amplifying the cDNA using the first and the second pluralities of oligonucleotide primers to produce first and second amplification products;
sequencing the first and the second amplification products to produce sequencing reads;
quantifying the sequencing reads; and
determining, based on the quantity of sequencing reads, a presence or absence of dengue virus RNA in the sample.

19. The method of claim 18, wherein the sample is a biological sample.

20. The method of claim 19, wherein the biological sample comprises blood or serum.

21. The method of claim 18, wherein the sample is an environmental sample.

22. The method of claim 21, wherein the environmental sample comprises an extract from one or more mosquitos, or a wastewater or air filter sample.

23. The method of claim 18, wherein the first plurality of oligonucleotide primers are configured for use as pairs, wherein the pairs are selected from the group consisting of SEQ ID NOs: 2 and 4; 5 and 8; 9 and 12; 13 and 16; 17 and 20; 21 and 24; 25 and 28; 29 and 32; 33 and 36; 37 and 40; 41 and 44; 45 and 48; 49 and 52; 53 and 56; 57 and 60; 61 and 64; 65 and 68; and 69 and 71; and
the second plurality of oligonucleotide primers are configured for use as pairs, wherein the pairs are selected from the group consisting of SEQ ID NOs: 3 and 6; 7 and 10; 11 and 14; 15 and 18; 19 and 22; 23 and 26; 27 and 30; 31 and 34; 35 and 38; 39 and 42; 43 and 46; 47 and 50; 51 and 54; 55 and 58; 59 and 62; 63 and 66; and 67 and 70.

24. The method of claim 18, wherein the sequencing is next generation sequencing (NGS).

25. The method of claim 18, further comprising assembling the sequencing reads to produce a consensus sequence.

26. The method of claim 25, wherein the consensus sequence is produced if at least 35 amplicons are detected in the sequencing reads.

27. A kit comprising:
one or more buffers;
a reverse transcriptase;
a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, and 71;
a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, and 70;
a DNA polymerase; and
one or more library preparation agents.


SEQUENCE LISTING (141 sequences)

SEQ ID NO: 1 - RNA, 10736 nt, Dengue virus
  source 1..10736: mol_type = other RNA
anttgttagtctnngtggaccgacaagaacagtttcgaatcggaagcttgcttaacgtagttctgacagttttttatttagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacccccaacagcaggaattttggctagatggggctcattcaagaagaatggagcgattaaagtgctacggggtttcaagaaagaaatctcaaacatgctgagcataatgaatagaagaaaaagatccgtgaccatgctccttatgctgctgcccacagccctggcgttccatctgaccacacgagggggagagccgcacatgatagttagcaagcaggaaagaggaaagtcacttttgtttaagacctctgcaggtgtcaacatgtgcacccttattgcgatggatttgggagagttatgtgaggacacaatgacttacaaatgcccccggatcactgaggcggaaccagatgacgttgactgttggtgcaatgccacggacacatgggtgacctatggaacgtgttctcaaactggcgaacaccgacgagacaagcgttccgtcgcactggccccacatgtggggcttggtctagaaacaagagccgaaacgtggatgtcctctgaaggcgcttggaaacaaatacaaaaagtggagacttgggctctgagacacccaggattcacggtaacagccctctttctagcacatgccataggaacatccatcacccagaaagggattattttcattttgttgatgctggtaacaccatccatggccatgcgatgcgtgggaataggcaacagagacttcgtggaaggactgtcaggagcaacgtgggtggatgtggtactggagcatggaagttgcgtcaccaccatggcaaaagataaaccaacattggacattgaactcttgaagacggaggtcacaaaccctgccgtcctgcgtaaactgtgcattgaagctaaaatatcaaacaccaccaccgattcaagatgtccaacacaaggggaagccacactggtggaagaacaagacgcgaacttcgtgtgtcgacgaacgtttgtggacagaggctggggcaatggctgtgggcttttcggaaaaggtagcctaataacgtgtgctaagttcaagtgtgtgacaaaactggaaggaaagattgttcaatatgagaacttgaaatattcagtgatagtcaccgtccacactggtgaccagcaccaggtgggaaatgagaccacagaacatggaacaactgcaaccataacacctcaagctcctacgtcggaaatacagctgaccgactacggagctcttacattggattgctcacccagaacagggctagactttaatgagatggtgttgttgacaatgaaagaaaaatcatggcttgtccacaaacaatggtttctagacttaccactgccctggacctcgggagcttcaacaccccaagagacttggaacagacaagatttgctggttacatttaagacagctcatgcaaagaagcaggaagtagtcgtactaggatcacaagaaggagcaatgcacactgcgttgaccggagcgacagaaatccaarcgtctggaacgacaacaatttttgcaggacacttgaaatgtagactaaaaatggacaaactgaccttaaaagggatgtcatatgtgatgtgcacaggctcattcaagttagagaaagaagtggctgagacccagcatggaactgttctagtgcaggttaaatacgaaggaacagatgcaccatgcaagatccccttttcgacccaagatgagaaaggagtaacccagaatgggagattgataacagccaaccccatagtcactgacaaagaaaaaccagtcaacattgaggcagaaccaccttttggtgagagttacatcgtggtaggagcaggtgaaaaagctttgaaactaagctggttcaagaaaggaagcagcatagggaaaatgcttgaagcaactgcccgaggagcacgaaggatggccatcctaggagacaccgcatgggacttcggttctataggaggagtgttcacgtctgtgggaaaactggtacaccagatttttggaactgcatatggagttttgttcagcggtgtttcctggactatgaaaataggaatagggattctgctgacatggctaggattaaattcaaggagcacgtccctttcgatgacgtgcattgcagttggcatggttacactgtacctaggagtcatggttcaggcggactcgggatgtgtaattaactggaaaggcagagaactcaaatgtggaagtggcatttttgtcaccaatgaagttcacacttggacagagcaatacaaattccaggccgactccccaaagagactatcagcagccattgggaaggcatgggaagagggtgtgtgtggaattcgatcagccactcgtctcgagaacatcatgtggaagcagatatcaaatgaactaaaccacatcttacttgaaaatgacatgaaattcacagtggtcgtaggagatgttagtgggatcttggcccaaggaaaaaaaatgattgggccacaacccatggaacacaaatactcgtggaaaagctggggaaaagccaaaatcataggagcagatgtacagaacaccaccttcattatcgacggcccaaacaccccagaatgccctgatgaccaaagagcatggaacatttgggaagttgaggactatggatttggaattttcacgacaaatatatggttgaaattgcgtgactcctacacccaagtgtgtgacccccggctaatgtcagctgccatcaaggacagcaaggcagttcatgccgatatggggtactggatagaaagtgaaaagaacgagacctggaagctggcgagagcctccttcatagaagttaagacatgcgtctggccaaaatcccacactctatggagcaatggagttttggaaagtgaaatgataatcccaaagatatatggaggaccaatatctcagcacaactacagaccaggatatttcacacaaacagcagggccgtggcacctaggcaagttggaactagattttgatttgtgtgagggtaccacagttgttgtggatgaacattgtggaaatcgaggaccatctcttagaaccacaacagtcacaggaaagataatccatgaatggtgctgtagatcttgtacgctaccccccttacgtttcaaaggagaagacgggtgttggtacggtatggaaatcagaccagtcaaggacaaggaagagaacctagttaagtcattggtctctgcagggtcaggagaagtggatagcttttcactaggactgctatgcatatcaataatgatcgaagaggtgatgagatccagatggagtagaaaaatgctgatgactggaacactggctgtgttcctccttctcataatgggacaattgacatggaatgatctgatcaggttatgcatcatggttggagccaatgcttcagacaggatggggatgggaacaacgtacctagctctgatggccacttttaaaatgagaccaatgttcgctgtcgggttattatttcgcagactaacatctagagaagttcttcttcttacgattggattgagtctggtggcatctgtggagctaccaaattccttggaggagctgggggatggacttgcaatgggcatcatgattttaaaattactgactgactttcagtcacatcagctgtgggctgccctgctgtccttgacatttatcaaaacaactttttcattgcactatgcatggaagacaatggctatggtactgtcaattgtatctctcttccctttatgcctgtccacgacctctcaaaaaacaacatggcttccggtgctgttgggatctcttggatgcaaaccactaaccatgtttcttatagcagaaaacaaaatctggggaaggaaaagttggcccctcaatgaaggaatcatggctgttggaatagttagcatcctactaagttcactcctcaagaatgatgtgccgctagccggcccactaatagctggaggtatgctaatagcatgttatgttatatccggaagctcagccgatttatcactggagaaagcggctgaggtctcctgggaagaagaagcagaacactctggtgcctcacacaacatactagtggaagtccaagatgatggaaccatgaagataaaagatgaagagagagatgacacactcaccattctccttaaagcaactctgttggcagtctcaggggtgtacccaatatcaataccagcgaccctttttgtgtggtatttttggcagaaaaagaaacagagatcaggagtgctatgggacacacctagccctccagaagtggaaagagcagttcttgatgatggtatctatagaattctgcaaagaggactgttgggcaggtcccaagtaggagtaggagttttccaagaaggcgtgttccacacaatgtggcatgtcaccaggggagctgtcctcatgtatcaagggaagagactggaaccgagttgggccagtgtcaaaaaagacttgatctcatatggaggaggttggaggtttcaaggatcctggaacacgggagaagaagtgcaggtgattgctgttgaaccggggaaaaaccccaaaaacgtacagacagcgccgggcaccttcaagacccctgaaggcgaagttggagccatagccctagattttaaacccggcacatctggatctcccattgtgaacagagagggaaaaatagtaggtctttatggaaatggagtggtgacaacaagtggaacctacgtcagtgccatagctcaagctaaagcatcacaagaagggcctctaccagagattgaggacgaggtgtttaggaaaagaaacttaacaataatggacctacatccaggatcggggaaaacaagaagatatcttccagccatagtccgtgaggctataaaaaggaagctgcgtacgctaatcttggctcccacaagagttgtcgcttctgaaatggcagaggcgctcaagggaatgccaataaggtatcagacaacagcagtgaagagtgaacacacaggaaaggagatagttgaccttatgtgccatgccactttcaccatgcgtctcctgtctcccgtgagagttcccaattacaacatgattatcatggatgaagcacatttcaccgatccagccagcatagcggccagagggtacatctcaacccgggtgggcatgggtgaagcagctgcgatcttcatgacagccactcccccaggatcggtggaggcctttccacagagcaatgcagttatccaagatgaggaaagagacattcctgagagatcatggaactcaggctatgactggatcactgatttcccaggtaaaacagtctggtttgttccaagcattaaatcaggaaatgacattgccaactgtttaagaaagaatgggaaacgggtgatccaattgagcagaaaaacctttgatactgagtaccagaaaacaaaaaataatgactgggactatgtcgtcacaacagacatttccgaaatgggagcaaacttccgagccgacagggtaatagacccaagacggtgtttgaaaccggtaatactaaaagatggtccagagcgtgtcattctagccggaccgatgccagtgactgtggccagtgccgcccagaggagaggaagaattggaaggaaccaaaataaggaaggtgatcagtacatttacatgggacagccttyaaacaacgatgaggatcacgctcattggacagaagcaaaaatgctccttgacaacataaacacaccagaagggattatcccagccctctttgagccggagagagraaaaagtgcagcaatagacggggaatacagactgcggggtgaagcaaggaaaacgttcgtggagctcatgagaagaggagatctacctgtctggctatcctacaaagttgcctcagaaggcttccagtactctgacagaaagtggtgctttgatggggaaaggaacaaccaggtgttggaggagaacatggacgtggagatctggacaaaagaaggagaaagaaagaaactacgaccccgctggctggacgccagaacatactctgacccacaggctctgcgcgagtttaaagagtttgcagcaggaagaagaagcgtctcaggtgatctaatattagaaatagggaaacttccacaacatttgacgcaaagggcccagaatgctttggacaacctggtcatgttgcacaactccgaacaaggaggaaaagcctatagacatgctatggaagaactaccagacaccatagaaacgttgatgctcctagctttgatagctgtgttaactggtggagtgacgctgttcttcctatcaggaagaggcctagggaaaacatctatcggcctactctgcgtgatggcttcaagcgtactgttatgggtggccagtgtggagccccattggatagcggcctccatcatactggagttctttctgatggtgctgcttattccagagccagacagacaacgcactccacaggacaaccagctggcatatgtggtgataggtttgttattcatgatattgacagtagcagccaatgagatgggattattggaaaccacaaagaaagacctagggattggccatgtggctgttgaaaaccaccaccatgccacaatgctggacgtagacttacrtccagcttcagcctggaccctctatgcagtggccacaacaattatcactcccatgatgagacacacaattgaaaacacaacggcaaatatttccctgacagctattgcaaaccaggcagctatattgatgggacttgacaagggatggccaatatcgaagatggacataggagttccactcctcgccttggggtgctattcccaggtgaacccgctgacgctgatagcggcggtattgatgctagtggctcattacgccataattggacctggactgcaagcaaaagccactagagaagctcaaaaaagaacagcggccggaataatgaaaaatccaactgtcgacggaattgttgcaatagatttggaccctgtggtttatgatgcaaaatttgaaaaacagctaggccaaataatgttgttgatactttgcacatcacagattctcttgatgcggactacatgggccttgtgtgaatccatcacactggctactggacctctgaccacgctttgggagggatctccaggaaaattctggaacaccacgatcgcggtgtccatggcaaacattttcaggggaagttatctagcaggagcaggtctggccttctcattaatgaaatctctaggaggaggtaggagaggtacgggagcccaaggggaaacactgggagaaaaatggaaaagacagctaaaccaactgagcaagtcagaattcaacacttacaaaaggagtgggattatggaggtggatagatctgaagctaaagagggactgaaaagaggagaaacaaccaaacatgcagtgtcgagaggaacagccaaactgaggtggtttgtggagaggaaccttgtgaagccggaagggaaagtcatagacctcggttgtggaagaggtggctggtcatattattgtgctgggctgaagaaagtcacagaagtgaaaggatacacaaaaggaggacctggacatgaggaaccaatcccaatggcgacctatggatggaacctagtaaagctacactccgggaaagatgtattctttacaccacctgagaaatgtgacacccttttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtggaaactctggagcaaatgcaaagaaaacatggaggaatgctagtgcgaaatccactctcaagaaattccacccatgaaatgtactgggtttcatgtggaacaggaaacattgtgtcagcagtaaacatgacatctagaatgttgctaaatcggttcacaatggctcacaggaagccaacatatgaaagagacgtggacttaggtgctggaacaagacatgtggcagtggaaccagaggtagccaacctagatatcattggccagaggatagagaatataaaaaatgagcataagtcaacatggcattatgatgaggacaatccatacaaaacatgggcctatcatggatcatatgaggttaagccatcaggatcagcctcatccatggtcaatggcgtggtgagattgctcaccaaaccatgggatgttatccctatggtcacacaaatagccatgactgacactacaccctttggacaacagagggtgtttaaagagaaagttgacacgcgcacaccaaaagcaaaacgaggcacagcacaaatcatggaggtgacagccaggtggttatggggttttctttctagaaacaaaaaaccaagaatctgcacaagagaggagttcacaagaaaagtcaggtcaaacgcagctattggagcagtgttcgttgatgaaaatcaatggaactcagcaaaagaagcggtggaagatgaacggttctgggaccttgtgcacagagagagggagctccataaacagggaaaatgtgccacgtgtgtttacaatatgatggggaagagagagaaaaaattaggagagttcggaaaggcaaaaggaagtcgtgcaatatggtacatgtggttgggagcacgctttctagagttcgaagcccttggtttcatgaacgaagatcactggttcagtagagagaattcactcagtggagtggaaggagaaggactccacaaacttggatatatactcagagacatatcaaagattccagggggaaatatgtatgcagatgacacagccggatgggacacaagaataacagaggatgatcttcagaatgaggccaaaatcactgacatcatggagcccgaacatgccctattggctacgtcaatctttaagctgacctaccaaaacaaggtggtaagggtgcagagaccagcaaaaaatggaaccgtgatggatgtcatatccagacgtgaccagagaggaagtggacaggtcggaacttatggcttaaacactttcactaacatggaggcccaactaataagacaaatggagtctgagggaatcttttcacccagcgaattggagaccccaaatttagccgaaagagttctcgactggttggaaaaacatggcgtcgaaaggctgaaaagaatggcaatcagcggagatgactgtgtggtgaaaccaactgatgacaggttcgcaacagccttaacagctttgaatgacatgggaaaagtaagaaaagacataccgcaatgggaaccttcaaaaggatggaatgattggcaacaagtgcctttttgttcacaccatttccaccagctgatcatgaaggatgggagggagatagtggtgccatgccgcaaccaagatgaacttgtgggtagggctagagtatcacaaggcgccggatggagcctgagagaaactgcatgcctaggcaagtcatatgcacaaatgtggcagctgatgtacttccacaggagagacctgagactagcggccaatgccatctgttcagccgttccaattgattgggtcccaaccagccgcaccacctggtcgatccatgcccatcatcaatggatgacaacagaagacatgttgtcagtgtggaatagggtttggatagaggaaaacccatggatggaggataaaacccatgtatccagttgggaagatgttccatacttaggaaaaagggaagatcagtggtgtggatccctgataggcttaacagcaagggccacctgggccactaatatacaagtggccataaaccaagtgagaaggcttattgggaatgagaattatctagattacatgacatcaatgaagagattcaagaatgagagtgatcccgaaggggcactctggtaagtcaacacattcacaaaacaaaggaaaataagaaatcaaacaaggcaagaagtcaggccggattaagccatagtacggtaagagctatgctgcctgtgagccccgtctaaggacgtaaaatgaagtcaggccggaagccacggtttgagcaaaccgtgctgcctgtagctccatcgtggggatgtaaaaacccgggaggctgcaacccatggaagctgtacgcatggggtagcagactagtggttagaggagacccctcccaaaacacaacgcagcagcggggcccaacaccaggggaagctgtaccctggtggtaaggactagaggttagaggagaccccccgcacaacaataaacagcatattgacgctgggagagaccagagatcctgctgtctctacagcatcattccaggcacagaacgccagaaaatggaatggtgctgttgaatcaacaggttct

SEQ ID NO: 2 - DNA, 22 bp, synthetic construct
  source 1..22: mol_type = other DNA
ggaccgacaagaacagtttcga

SEQ ID NO: 3 - DNA, 22 bp, synthetic construct
  source 1..22: mol_type = other DNA
tggagcgattaaagtgctacgg

SEQ ID NO: 4 - DNA, 22 bp, synthetic construct
  source 1..22: mol_type = other DNA
ggagcatggtcacggatctttt

SEQ ID NO: 5 - DNA, 22 bp, synthetic construct
  source 1..22: mol_type = other DNA
ccagatgacgttgactgttggt

SEQ ID NO: 6 - DNA, 20 bp, synthetic construct
  source 1..20: mol_type = other DNA
aacgcttgtctcgtcggtgt

SEQ ID NO: 7 - DNA, 22 bp, synthetic construct
  source 1..22: mol_type = other DNA
cgtgggaataggcaacagagac

SEQ ID NO: 8 - DNA, 22 bp, synthetic construct
  source 1..22: mol_type = other DNA
aacttccatgctccagtaccac

SEQ ID NO: 9 - DNA, 22 bp, synthetic construct
  source 1..22: mol_type = other DNA
tgtgggcttttcggaaaaggta

SEQ ID NO: 10 - DNA, 28 bp, synthetic construct
  source 1..28: mol_type = other DNA
tctcatattgaacaatctttccttccag

SEQ ID NO: 11 - DNA, 22 bp, synthetic construct
  source 1..22: mol_type = other DNA
tggcttgtccacaaacaatggt

SEQ ID NO: 12 - DNA, 22 bp, synthetic construct
  source 1..22: mol_type = other DNA
gtctgttccaagtctcttgggg

SEQ ID NO: 13 - DNA, 22 bp, synthetic construct
  source 1..22: mol_type = other DNA
gaaagaagtggctgagacccag

SEQ ID NO: 14 - DNA, 22 bp, synthetic construct
  source 1..22: mol_type = other DNA
tcgaaaaggggatcttgcatgg

SEQ ID NO: 15 - DNA, 20 bp, synthetic construct
  source 1..20: mol_type = other DNA
ttgaagcaactgcccgagga

SEQ ID NO: 16 - DNA, 24 bp, synthetic construct
  source 1..24: mol_type = other DNA
gcagttccaaaaatctggtgtacc

SEQ ID NO: 17 - DNA, 23 bp, synthetic construct
  source 1..23: mol_type = other DNA
agaactcaaatgtggaagtggca

SEQ ID NO: 18 - DNA, 22 bp, synthetic construct
  source 1..22: mol_type = other DNA
ggagtcggcctggaatttgtat

SEQ ID NO: 19 - DNA, 22 bp, synthetic construct
  source 1..22: mol_type = other DNA
aatactcgtggaaaagctgggg

SEQ ID NO: 20 - DNA, 21 bp, synthetic construct
  source 1..21: mol_type = other DNA
ggtgtttgggccgtcgataat

SEQ ID NO: 21 - DNA, 23 bp, synthetic construct
  source 1..23: mol_type = other DNA
cgagagcctccttcatagaagtt

SEQ ID NO: 22 - DNA, 27 bp, synthetic construct
  source 1..27: mol_type = other DNA
tctttgggattatcatttcactttcca

SEQ ID NO: 23 - DNA, 22 bp, synthetic construct
  source 1..22: mol_type = other DNA
cccccttacgtttcaaaggaga

SEQ ID NO: 24 - DNA, 26 bp, synthetic construct
  source 1..26: mol_type = other DNA
acttaactaggttctcttccttgtcc

SEQ ID NO: 25 - DNA, 22 bp, synthetic construct
  source 1..22: mol_type = other DNA
tgggaacaacgtacctagctct

SEQ ID NO: 26 - DNA, 25 bp, synthetic construct
  source 1..25: mol_type = other DNA
tgttagtctgcgaaataataacccg

SEQ ID NO: 27 - DNA, 22 bp, synthetic construct
  source 1..22: mol_type = other DNA
tcttccctttatgcctgtccac

SEQ ID NO: 28 - DNA, 22 bp, synthetic construct
  source 1..22: mol_type = other DNA
tggtttgcatccaagagatccc

SEQ ID NO: 29 - DNA, 22 bp, synthetic construct
  source 1..22: mol_type = other DNA
ctgaggtctcctgggaagaaga

SEQ ID NO: 30 - DNA, 28 bp, synthetic construct
  source 1..28: mol_type = other DNA
ctcttcatcttttatcttcatggttcca

SEQ ID NO: 31 - DNA, 22 bp, synthetic construct
  source 1..22: mol_type = other DNA
ggtcccaagtaggagtaggagt

SEQ ID NO: 32 - DNA, 22 bp, synthetic construct
  source 1..22: mol_type = other DNA
gatacatgaggacagctcccct

SEQ ID NO: 33 - DNA, 22 bp, synthetic construct
  source 1..22: mol_type = other DNA
tcccattgtgaacagagaggga

SEQ ID NO: 34 - DNA, 22 bp, synthetic construct
  source 1..22: mol_type = other DNA
gctatggcactgacgtaggttc

SEQ ID NO: 35 - DNA, 22 bp, synthetic construct
  source 1..22: mol_type = other DNA
acagcagtgaagagtgaacaca

SEQ ID NO: 36 - DNA, 22 bp, synthetic construct
  source 1..22: mol_type = other DNA
gacaggagacgcatggtgaaag

SEQ ID NO: 37 - DNA, 22 bp, synthetic construct
  source 1..22: mol_type = other DNA
tggaactcaggctatgactgga

SEQ ID NO: 38 - DNA, 23 bp, synthetic construct
  source 1..23: mol_type = other DNA
acagttggcaatgtcatttcctg

SEQ ID NO: 39 - DNA, 21 bp, synthetic construct
  source 1..21: mol_type = other DNA
tgtcattctagccggaccgat

SEQ ID NO: 40 - DNA, 24 bp, synthetic construct
  source 1..24: mol_type = other DNA
aggctgtcccatgtaaatgtactg

SEQ ID NO: 41 - DNA, 22 bp, synthetic construct
  source 1..22: mol_type = other DNA
cctcagaaggcttccagtactc

SEQ ID NO: 42 - DNA, 22 bp, synthetic construct
  source 1..22: mol_type = other DNA
ccatgttctcctccaacacctg

SEQ ID NO: 43 - DNA, 26 bp, synthetic construct
  source 1..26: mol_type = other DNA
aggaaaagcctatagacatgctatgg

SEQ ID NO: 44 - DNA, 23 bp, synthetic construct
  source 1..23: mol_type = other DNA
tccaccagttaacacagctatca

SEQ ID NO: 45 - DNA, 22 bp, synthetic construct
  source 1..22: mol_type = other DNA
acagtagcagccaatgagatgg

SEQ ID NO: 46 - DNA, 22 bp, synthetic construct
  source 1..22: mol_type = other DNA
gtggttttcaacagccacatgg

SEQ ID NO: 47 - DNA, 24 bp, synthetic construct
  source 1..24: mol_type = other DNA
cgaagatggacataggagttccac

SEQ ID NO: 48 - DNA, 22 bp, synthetic construct
  source 1..22: mol_type = other DNA
gcttgcagtccaggtccaatta

SEQ ID NO: 49 - DNA, 22 bp, synthetic construct
  source 1..22: mol_type = other DNA
atgggccttgtgtgaatccatc

SEQ ID NO: 50 - DNA, 22 bp, synthetic construct
  source 1..22: mol_type = other DNA
gcgatcgtggtgttccagaatt

SEQ ID NO: 51 - DNA, 23 bp, synthetic construct
  source 1..23: mol_type = other DNA
agggactgaaaagaggagaaaca

SEQ ID NO: 52 - DNA, 22 bp, synthetic construct
  source 1..22: mol_type = other DNA
gcttcacaaggttcctctccac

SEQ ID NO: 53 - DNA, 22 bp, synthetic construct
  source 1..22: mol_type = other DNA
gagtcctctccgaacccaacta

SEQ ID NO: 54 - DNA, 22 bp, synthetic construct
  source 1..22: mol_type = other DNA
attggtttcctctgagccatgg

SEQ ID NO: 55 - DNA, 22 bp, synthetic construct
  source 1..22: mol_type = other DNA
aagagacgtggacttaggtgct

SEQ ID NO: 56 - DNA, 24 bp, synthetic construct
  source 1..24: mol_type = other DNA
tcctctggccaatgatatctaggt

SEQ ID NO: 57 - DNA, 22 bp, synthetic construct
  source 1..22: mol_type = other DNA
aaagagaaagttgacacgcgca

SEQ ID NO: 58 - DNA, 22 bp, synthetic construct
  source 1..22: mol_type = other DNA
agaaaaccccataaccacctgg

SEQ ID NO: 59 - DNA, 22 bp, synthetic construct
  source 1..22: mol_type = other DNA
agagttcggaaaggcaaaagga

SEQ ID NO: 60 - DNA, 22 bp, synthetic construct
  source 1..22: mol_type = other DNA
accaagggcttcgaactctaga

SEQ ID NO: 61 - DNA, 24 bp, synthetic construct
  source 1..24: mol_type = other DNA
tggctacgtcaatctttaagctga

SEQ ID NO: 62 - DNA, 22 bp, synthetic construct
  source 1..22: mol_type = other DNA
tgacatccatcacggttccatt

SEQ ID NO: 63 - DNA, 22 bp, synthetic construct
  source 1..22: mol_type = other DNA
aactgatgacaggttcgcaaca

SEQ ID NO: 64 - DNA, 22 bp, synthetic construct
  source 1..22: mol_type = other DNA
tgaaggttcccattgcggtatg

SEQ ID NO: 65 - DNA, 20 bp, synthetic construct
  source 1..20: mol_type = other DNA
acctgagactagcggccaat

SEQ ID NO: 66 - DNA, 22 bp, synthetic construct
  source 1..22: mol_type = other DNA
ttgatgatgggcatggatcgac

SEQ ID NO: 67 - DNA, 30 bp, synthetic construct
  source 1..30: mol_type = other DNA
gggaatgagaattatctagattacatgaca

SEQ ID NO: 68 - DNA, 22 bp, synthetic construct
  source 1..22: mol_type = other DNA
tgtgttgacttaccagagtgcc

SEQ ID NO: 69 - DNA, 22 bp, synthetic construct
  source 1..22: mol_type = other DNA
ggccggattaagccatagtacg

SEQ ID NO: 70 - DNA, 24 bp, synthetic construct
  source 1..24: mol_type = other DNA
tctaacctctagtccttaccacca

SEQ ID NO: 71 - DNA, 22 bp, synthetic construct
  source 1..22: mol_type = other DNA
cagcaccattccattttctggc

SEQ ID NO: 72 - DNA, 20 bp, synthetic construct
  source 1..20: mol_type = other DNA
acgtggaccgacaagaacag

SEQ ID NO: 73 - DNA, 20 bp, synthetic construct
  source 1..20: mol_type = other DNA
actatcatgtgcggctctcc

SEQ ID NO: 74 - DNA, 20 bp, synthetic construct
  source 1..20: mol_type = other DNA
tgaccatgctccttatgctg

SEQ ID NO: 75 - DNA, 20 bp, synthetic construct
  source 1..20: mol_type = other DNA
caagcgccttcagaggacat

SEQ ID NO: 76 - DNA, 20 bp, synthetic construct
  source 1..20: mol_type = other DNA
caccgacgagacaaacgttc

SEQ ID NO: 77 - DNA, 21 bp, synthetic construct
  source 1..21: mol_type = other DNA
agggtttgtgacttccgtctt

SEQ ID NO: 78 - DNA, 20 bp, synthetic construct
  source 1..20: mol_type = other DNA
ggaagttgcgtcaccaccat

SEQ ID NO: 79 - DNA, 23 bp, synthetic construct
  source 1..23: mol_type = other DNA
ccatgttctgtgctttcatttcc

SEQ ID NO: 80 - DNA, 29 bp, synthetic construct
  source 1..29: mol_type = other DNA
aaactggaaggaaagatagttcaatatga

SEQ ID NO: 81 - DNA, 22 bp, synthetic construct
  source 1..22: mol_type = other DNA
agtgtgcattgctccttcttgt

SEQ ID NO: 82 - DNA, 22 bp, synthetic construct
  source 1..22: mol_type = other DNA
tgctggtgacatttaagacagc

SEQ ID NO: 83 - DNA, 25 bp, synthetic construct
  source 1..25: mol_type = other DNA
tgttgactggtttttctttgtcagt

SEQ ID NO: 84 - DNA, 21 bp, synthetic construct
  source 1..21: mol_type = other DNA
gaaggaacagatgcaccatgc

SEQ ID NO: 85 - DNA, 24 bp, synthetic construct
  source 1..24: mol_type = other DNA
ccatgtcagcagaacccctattcc

SEQ ID NO: 86 - DNA, 23 bp, synthetic construct
  source 1..23: mol_type = other DNA
acaccagatttttggaactgcat

SEQ ID NO: 87 - DNA, 28 bp, synthetic construct
  source 1..28: mol_type = other DNA
tcatttgatatttgcttccacatgatgt

SEQ ID NO: 88 - DNA, 23 bp, synthetic construct
  source 1..23: mol_type = other DNA
tgaagttcacacttggacagagc

SEQ ID NO: 89 - DNA, 23 bp, synthetic construct
  source 1..23: mol_type = other DNA
acttcccaaatgttccatgctct

SEQ ID NO: 90 - DNA, 22 bp, synthetic construct
  source 1..22: mol_type = other DNA
gaaaagctggggaaaggctaaa

SEQ ID NO: 91 - DNA, 22 bp, synthetic construct
  source 1..22: mol_type = other DNA
tgtgctgagatattggtcctcc

SEQ ID NO: 92 - DNA, 22 bp, synthetic construct
  source 1..22: mol_type = other DNA
cccacactctatggagcaatgg

SEQ ID NO: 93 - DNA, 20 bp, synthetic construct
  source 1..20: mol_type = other DNA
acttcccctgaccctgcaga

SEQ ID NO: 94 - DNA, 26 bp, synthetic construct
  source 1..26: mol_type = other DNA
acaggaaagataattcatgaatggtg

SEQ ID NO: 95 - DNA, 26 bp, synthetic construct
  source 1..26: mol_type = other DNA
aacattggtctcattttaaaagtggc

SEQ ID NO: 96 - DNA, 22 bp, synthetic construct
  source 1..22: mol_type = other DNA
tatgcatcatggttggagccaa

SEQ ID NO: 97 - DNA, 21 bp, synthetic construct
  source 1..21: mol_type = other DNA
tttttgggaggtcgtggacag

SEQ ID NO: 98 - DNA, 25 bp, synthetic construct
  source 1..25: mol_type = other DNA
gcaatgggcattatgattttaaaat

SEQ ID NO: 99 - DNA, 22 bp, synthetic construct
  source 1..22: mol_type = other DNA
gctattagcatgcctccagcta

SEQ ID NO: 100 - DNA, 22 bp, synthetic construct
  source 1..22: mol_type = other DNA
atctggggaaggaaaagttggc

SEQ ID NO: 101 - DNA, 25 bp, synthetic construct
  source 1..25: mol_type = other DNA
ccagatctctgtttctttttctgcc

SEQ ID NO: 102 - DNA, 29 bp, synthetic construct
  source 1..29: mol_type = other DNA
aagataaaggatgaagagagagatgacac

SEQ ID NO: 103 - DNA, 22 bp, synthetic construct
  source 1..22: mol_type = other DNA
tcttctcccgtgttccaggatc

SEQ ID NO: 104 - DNA, 22 bp, synthetic construct
  source 1..22: mol_type = other DNA
tgtatcaagggaagagactgga

SEQ ID NO: 105 - DNA, 25 bp, synthetic construct
  source 1..25: mol_type = other DNA
atcctggatgtaggtccattattgt

SEQ ID NO: 106 - DNA, 23 bp, synthetic construct
  source 1..23: mol_type = other DNA
gctcaagctaaagcatcacaaga

SEQ ID NO: 107 - DNA, 20 bp, synthetic construct
  source 1..20: mol_type = other DNA
taccctctggccgctatgct

SEQ ID NO: 108 - DNA, 20 bp, synthetic construct
  source 1..20: mol_type = other DNA
tctcccgtgagagttcccaa

SEQ ID NO: 109 - DNA, 22 bp, synthetic construct
  source 1..22: mol_type = other DNA
gtgacgacgtagtcccagtcgt

SEQ ID NO: 110 - DNA, 22 bp, synthetic construct
  source 1..22: mol_type = other DNA
aaacgggtgattcaattgagca

SEQ ID NO: 111 - DNA, 22 bp, synthetic construct
  source 1..22: mol_type = other DNA
gctcgaagagggctgggataat

SEQ ID NO: 112 - DNA, 21 bp, synthetic construct
  source 1..21: mol_type = other DNA
cacgctcactggacagaagca

SEQ ID NO: 113 - DNA, 22 bp, synthetic construct
  source 1..22: mol_type = other DNA
tcttcttcctgctgcaaactct

SEQ ID NO: 114 - DNA, 22 bp, synthetic construct
  source 1..22: mol_type = other DNA
ctgacagaagatggtgctttga

SEQ ID NO: 115 - DNA, 21 bp, synthetic construct
  source 1..21: mol_type = other DNA
gtcactccaccagttaacaca

SEQ ID NO: 116 - DNA, 21 bp, synthetic construct
  source 1..21: mol_type = other DNA
tccgaacaaggaggaagagcc

SEQ ID NO: 117 - DNA, 22 bp, synthetic construct
  source 1..22: mol_type = other DNA
ccctaagtctttctttgtggtt

SEQ ID NO: 118 - DNA, 22 bp, synthetic construct
  source 1..22: mol_type = other DNA
gctggcatatgtggtgataggt

SEQ ID NO: 119 - DNA, 20 bp, synthetic construct
  source 1..20: mol_type = other DNA
catcaataccgccgctgtca

SEQ ID NO: 120 - DNA, 23 bp, synthetic construct
  source 1..23: mol_type = other DNA
ggacataggagttccacttctcg

SEQ ID NO: 121 - DNA, 23 bp, synthetic construct
  source 1..23: mol_type = other DNA
gctatcgtggtgttccaaaattt

SEQ ID NO: 122 - DNA, 22 bp, synthetic construct
  source 1..22: mol_type = other DNA
atgggccttgtgcgaatccatc

SEQ ID NO: 123 - DNA, 22 bp, synthetic construct
  source 1..22: mol_type = other DNA
ggtttcacaaggttcctctcca

SEQ ID NO: 124 - DNA, 23 bp, synthetic construct
  source 1..23: mol_type = other DNA
aagaggagaaacaaccaaacatg

SEQ ID NO: 125 - DNA, 22 bp, synthetic construct
  source 1..22: mol_type = other DNA
tttcctctgagccatggttcca

SEQ ID NO: 126 - DNA, 24 bp, synthetic construct
  source 1..24: mol_type = other DNA
gtgtgatattggtgagtcctctcc

SEQ ID NO: 127 - DNA, 24 bp, synthetic construct
  source 1..24: mol_type = other DNA
tctggccaatgatatctaggttgg

SEQ ID NO: 128 - DNA, 20 bp, synthetic construct
  source 1..20: mol_type = other DNA
acaatggctcacaggaaacc

SEQ ID NO: 129 - DNA, 20 bp, synthetic construct
  source 1..20: mol_type = other DNA
cacttggctgtcacctccat

SEQ ID NO: 130 - DNA, 22 bp, synthetic construct
  source 1..22: mol_type = other DNA
ctacaccctttggacaacagag

SEQ ID NO: 131 - DNA, 22 bp, synthetic construct
  source 1..22: mol_type = other DNA
gctcccaaccacatgtaccata

SEQ ID NO: 132 - DNA, 23 bp, synthetic construct
  source 1..23: mol_type = other DNA
tgtgtttacaacatgatggggaa

SEQ ID NO: 133 - DNA, 21 bp, synthetic construct
  source 1..21: mol_type = other DNA
tttttgctggtctctgtaccc

SEQ ID NO: 134 - DNA, 24 bp, synthetic construct
  source 1..24: mol_type = other DNA
agaatgaggccaaaattactgaca

SEQ ID NO: 135 - DNA, 22 bp, synthetic construct
  source 1..22: mol_type = other DNA
tcagagctgttaaggctgttgc

SEQ ID NO: 136 - DNA, 22 bp, synthetic construct
  source 1..22: mol_type = other DNA
tgaaaagaatggcaatcagcgg

SEQ ID NO: 137 - DNA, 21 bp, synthetic construct
  source 1..21: mol_type = other DNA
aactggaacggctgaacagat

SEQ ID NO: 138 - DNA, 22 bp, synthetic construct
  source 1..22: mol_type = other DNA
tatgcacaaatgtggcagctga

SEQ ID NO: 139 - DNA, 27 bp, synthetic construct
  source 1..27: mol_type = other DNA
tcttgaatctcttcattgatgtcatgt

SEQ ID NO: 140 - DNA, 21 bp, synthetic construct
  source 1..21: mol_type = other DNA
ctgggccaccaacatacaagt

SEQ ID NO: 141 - DNA, 22 bp, synthetic construct
  source 1..22: mol_type = other DNA
aggggtctcctctaaccactag